(19) United States
(12) Patent Application Publication
(10) Pub. No.: US 2025/0255816 A1
(43) Pub. Date: Aug. 14, 2025

(54) POLYSULFIDE MICROPARTICLES AND USES THEREOF

(71) Applicant: Vanderbilt University, Nashville, TN (US)

(72) Inventors: Craig L. Duvall, Nashville, TN (US); Carlisle R. DeJulius, Nashville, TN (US); Richard D'Arcy, Nashville, TN (US); Tonia S. Rex, Nashville, TN (US); Sarah Naguib, Nashville, TN (US)

(21) Appl. No.: 18/857,424

(22) PCT Filed: Apr. 18, 2023
(86) PCT No.: PCT/US2023/018982

§ 371 Date: Oct. 16, 2024

Related U.S. Application Data
(60) Provisional application No. 63/332,251, filed on Apr. 18, 2022.
(60) Provisional application No. 63/332,565, filed on Apr. 19, 2022.

(51) Int. Cl. A61K 9/16; A61K 31/713; A61P 27/06

(52) U.S. Cl. CPC A61K 9/1641; A61K 9/1682; A61K 31/713; A61P 27/06

(57) ABSTRACT

Disclosed herein are polysulfide microparticles that include a varying monomer composition. The monomer composition can control properties of the microparticles, such as crystallinity, which can aid in the production and stability of the microparticles. An example microparticle includes a polymer derived from monomers of propylene sulfide (PS) and ethylene sulfide (ES). The microparticles disclosed herein can be useful in drug delivery applications, such as treating inflammatory diseases. Also disclosed are methods of making the polysulfides and methods of making the microparticles.

### CROSS-REFERENCE TO RELATED APPLICATIONS

This application claims priority to U.S. Provisional Patent Application No. 63/332,251 filed on Apr. 18, 2022 and U.S. Provisional Patent Application No. 63/332,565 filed on Apr. 19, 2022, both of which are incorporated fully herein by reference.

### FEDERALLY SPONSORED RESEARCH

This invention was made with government support under grant numbers NBIB T-32-EB021937 and NEI R01 EY022349 awarded by the National Institutes of Health, and grant number DGE-1937963 awarded by the National Science Foundation under its Graduate Research Fellowship Program. The government has certain rights in the invention.

### TECHNICAL FIELD

This disclosure relates to polysulfide microparticles and their use in biomedical applications, such as drug delivery.

### BACKGROUND

Control of biomaterial formulation to produce, e.g., particles and other structures can be useful for drug delivery applications.

### SUMMARY

Embodiments herein relate to polysulfides and microparticles including the same. In one aspect, disclosed are microparticles comprising a polymer including recurring units of formula (I):

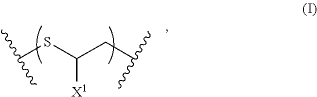

wherein X¹ is H or CH₃; the polymer includes recurring units of X¹ is CH₃ at about 1 mol % to about 70 mol % of the polymer; the polymer includes recurring units of X¹ is H at greater than or equal to 30 mol % of the polymer; and the microparticle has a diameter of greater than 5 μm.

In another aspect, disclosed are methods of treating an inflammatory disease in a subject in need thereof, the method comprising administering to the subject an effective amount of a microparticle as disclosed herein, optionally in combination with a pharmaceutically acceptable excipient.

In another aspect, disclosed are methods of making a polymer, the method comprising adding a first sulfide monomer and a second sulfide monomer to a reaction mixture via a syringe pump at less than or equal to 15 μl/minute, wherein the first sulfide monomer and the second sulfide monomer react to provide a random copolymer that is about 1% to about 40% crystalline as measured by X-ray diffractive spectroscopy.

In another aspect, disclosed are methods of making a microparticle, the method comprising adding a nanoparticle comprising a dextran or other polymer and a biologically active agent to a first solvent to provide a mixture, wherein the first solvent comprises a polymer including recurring units of formula (I) wherein X¹ is H or CH₃, the polymer includes recurring units of X¹ is CH₃ at about 1 mol % to about 70 mol % of the polymer, and the polymer includes recurring units of X¹ is H at greater than or equal to 30 mol % of the polymer; and adding a second solvent to the mixture and emulsifying the mixture to provide a microparticle comprising the polymer and the nanoparticle

### BRIEF DESCRIPTION OF THE DRAWINGS

FIG. 1 shows a schematic of an anionic ring-opening polymerization of propylene sulfide (PS) and ethylene sulfide (ES).

FIG. 2A-2B show characterization of poly(propylene sulfide-co-ethylene sulfide) (PPSES) library where E #denotes mol % of ES monomer. For example, E50 corresponds to 50 mol % of ES monomer. Gel permeation chromatography (GPC) shows unimodal molecular weight distributions (FIG. 2A), and differential scanning calorimetry (DSC) shows emergence of crystallization peaks at ES content >40% (arrows) (FIG. 2B).

FIG. 3 shows a schematic of the backbone oxidation of polysulfides to more hydrophilic polysulfoxides and polysulfones.

FIG. 4 shows a schematic of an oil-in-water (O/W) formulation.

FIG. 5 shows scanning electron microscopy (SEM) images of PPSES microparticles where E #denotes the mol % of ES monomer.

FIG. 6A-6B show optical microscopy demonstrating ROS-responsive degradation of E50 microparticles exposed to increasing concentrations of hydrogen peroxide (H₂O₂). FIG. 6A: Day 0-17. FIG. 6B: Day 24-41.

FIG. 7A-7D show E50 microparticles can be formulated in a wide range of size distributions visualized by SEM. FIG. 7A: SEM images of E50 microparticles homogenized at different rpm. FIG. 7B: bar graph showing particle diameter distribution for E50 microparticles homogenized at 5,000 rpm. FIG. 7C: bar graph showing particle diameter distribution for E50 microparticles homogenized at 700 rpm. FIG. 7D: bar graph showing particle diameter distribution for E50 microparticles homogenized at 600 rpm.

FIG. 8 shows a schematic of a solid-in-oil-in-water (S/O/W) formulation.

FIG. 9A-9B show SEM images of dextran precursor nanoparticles (FIG. 9A) and a bar graph showing the measurements of protein loading and encapsulation efficiency for nanoparticles containing 0 or 15% cationic DEA-dextran (FIG. 9B). BSA=bovine serum albumin and DEAE=diethylaminoethyl.

FIG. 10 shows a schematic of a study design for a mouse microbead occlusion model (MOM) of glaucoma and microparticle treatment.

FIG. 11 shows retinal reactive oxygen species (ROS) and erythropoietin (EPO) levels in mouse MOM model of glaucoma 6 weeks after microparticle injection.

FIG. 12A-12C show electroretinogram (ERG) and visual evoked potential (VEP) tests in mouse MOM model of glaucoma 6 weeks after microparticle injection. FIG. 12A: Retinal Ganglion Cell Function. FIG. 12B: Bipolar Cell Function. FIG. 12C: Signal Transmission to Visual Cortex. (*p<0.05, ** p<0.005, *** p<0.0005)

FIG. 13 shows example microparticle oxidation at 10 mM and 100 mM H₂O₂.

FIG. 14 shows endotoxin analysis for example microparticle formulations.

FIG. 15A-15B show characterization of example microparticles loaded with a cholesterol-modified oligonucleotide. FIG. 15A: SEM image of example microparticles loaded with an oligonucleotide. FIG. 15B: ROS-responsive release of oligonucleotide from example microparticles. Loss in fluorescence signal from the particles was used to indicate release of the fluoro-labeled oligonucleotide.

FIG. 16 shows an SEM image of ethylene glycol (EG) 18-modified oligonucleotide loaded into example microparticles.

FIG. 17 shows SEM images of example microparticles loaded with EPO at a high salt concentration (˜ 56,000 mOsm/kg).

FIG. 18 shows SEM images of example microparticles loaded with EPO at a low salt concentration (˜ 280-315 mOsm/kg).

FIG. 19 shows X-ray diffractive spectroscopy analysis for example polymers.

### DETAILED DESCRIPTION

Provided herein are microparticles that can be used for local, sustained drug delivery. The microparticles can provide at least two major advantages over currently used techniques: (1) size tunability, e.g., from 100s of nanometers to 100 s of micrometers in diameter, and (2) local ROS scavenging, providing the overall system anti-inflammatory properties. This is believed to be the first instance of a thioether-based microparticle system with both properties.

Traditional homopolymers of propylene sulfide (PS), poly(propylene sulfide) (PPS), can provide ROS scavenging, but cannot be formulated into microparticles with diameters greater than ˜5 μm due to the amorphous (non-crystalline) nature of PPS. This can reduce the local retention time and limit drug formulation strategies. It is likely, that both the potential to fabricate into a larger size and a greater physical stability of crystalline over amorphous microparticles may contribute to longer retention and sustained drug release for local therapy applications for the disclosed microparticles compared to PPS.

Further, in contrast to polysulfides, commonly used polyester systems (i.e. poly(lactic-co-glycolic acid), PLGA) have a wide range of formulation capabilities, including large microparticles, but break down into acidic degradation products, which can enhance local inflammation. Disclosed poly(propylene sulfide-co-ethylene sulfide) (PPSES) microparticles can exhibit both size tunability and can have localized anti-inflammatory effects due to the antioxidant properties of the thioether backbone (e.g., ROS-sequestering). Accordingly, the disclosed microparticles can have improved properties over currently used polyester-based systems.

### 1. DEFINITIONS

Unless otherwise defined, all technical and scientific terms used herein have the same meaning as commonly understood by one of ordinary skill in the art. In case of conflict, the present document, including definitions, will control. Example methods and materials are described below, although methods and materials similar or equivalent to those described herein can be used in practice or testing of the disclosed invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are contemplated, and for the range 1.5-2, the numbers 1.5, 1.6, 1.7, 1.8, 1.9, and 2 are contemplated.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group-O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylene,” as used herein, refers to a divalent group derived from a straight or branched chain hydrocarbon of 1 to 50 carbon atoms, for example, of 2 to 10 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “biologically active agent,” as used herein, refers to a substance that can act on a cell, virus, tissue, organ, organism, or the like, to create a change in the functioning of the cell, virus, tissue, organ, or organism. Examples of a biologically active agent include, but are not limited to, small molecule drugs, lipids, proteins, peptides, and nucleic acids. A biologically active agent is capable of treating and/or ameliorating a condition or disease, or one or more symptoms thereof, in a subject. Biologically active agents of the present disclosure also include prodrug forms of the agent.

The term “cyanoalkyl,” as used herein, means at least one —CN group is appended to the parent molecular moiety through an alkylene group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The term “effective dosage” or “therapeutic dosage” or “therapeutically effective amount” or “effective amount,” as used herein, refers to an amount sufficient to effect beneficial or desirable biological and/or clinical results to treat a disease or one or more of its symptoms and/or to prevent or reduce the risk of the occurrence or reoccurrence of the disease or disorder or symptom(s) thereof. A therapeutically effective amount is also one in which any toxic or detrimental effects of substance are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. In reference to inflammatory diseases an effective or therapeutically effective amount can include an amount sufficient to, among other things, decrease the underlying pathology associated with the inflammatory disease.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “oligonucleotide” refers to a polymer of nucleotides. The terms “polynucleotide,” “nucleic acid,” and “oligonucleotide”, may be used interchangeably herein. Typically, a polynucleotide comprises at least three nucleotides. Oligonucleotides can be single stranded or double stranded. Example oligonucleotides include, but are not limited to, DNA and RNA, such as mRNA, RNAi, siRNA, and shRNA.

A “protein” or “polypeptide” is a linked sequence of 50 or more amino acids linked by peptide bonds. A peptide is a linked sequence of 2 to 50 amino acids linked by peptide bonds. The polypeptide and peptide can be natural, synthetic, or a modification or combination of natural and synthetic. Proteins and polypeptides include proteins such as binding proteins, receptors, and antibodies. The terms “polypeptide,” and “protein” are used interchangeably herein.

The term “subject” includes humans and mammals (e.g., mice, rats, pigs, cats, dogs, and horses). Typical subjects of the present disclosure may include mammals, particularly primates, and especially humans. For veterinary applications, suitable subjects may include, for example, livestock such as cattle, sheep, goats, cows, swine, and the like; poultry such as chickens, ducks, geese, turkeys, and the like, as well as domesticated animals particularly pets such as dogs and cats. For research applications, suitable subjects may include mammals, such as rodents (e.g., mice, rats, hamsters), rabbits, primates, and swine such as inbred pigs and the like.

The term “treatment” or “treating,” as used herein, refers to protection of a subject from a disease, such as preventing, suppressing, repressing, ameliorating, or eliminating the disease. Preventing the disease involves administering a microparticle or pharmaceutical composition thereof of the present disclosure to a subject prior to onset of the disease. Suppressing the disease involves administering a microparticle or pharmaceutical composition thereof of the present disclosure to a subject after induction of the disease but before its clinical appearance. Repressing or ameliorating the disease involves administering a microparticle or pharmaceutical composition thereof of the present disclosure to a subject after clinical appearance of the disease.

### 2. MICROPARTICLES

Provided herein are microparticles that include a polysulfide polymer (also referred to herein as “the polymer”). It has been found by incorporating certain monomers at certain percentages into the polymer, the crystallinity of the polymer can be modulated. For example, the polymer may be derived from propylene sulfide and ethylene sulfide monomers, where the ethylene sulfide monomer can impart crystallinity to the polymer. Designing the polymer to have crystallinity can aid the polymer to be formulated into larger diameter particles (e.g., greater than 5 μm).

As discussed above, the polymer may have crystallinity, and as a result the microparticle may also have crystallinity. In some embodiments, the polymer, the microparticle, or both are semi-crystalline. In some embodiments, the polymer, the microparticle, or both have regions that are crystalline and regions that are amorphous. The polymer can have a crystallinity of about 1% to about 40% as measured by X-ray diffractive spectroscopy, such as about 1% to about 35%, about 1% to about 30%, about 1% to about 20%, about 1% to about 15%, about 1% to about 10%, about 1% to about 13%, about 2% to about 15%, about 3% to about 15%, about 5% to about 15%, or about 7% to about 13%. In some embodiments, the polymer has a crystallinity of greater than or equal to 1%, greater than or equal to 2%, greater than or equal to 3%, greater than or equal to 4%, greater than or equal to 5%, greater than or equal to 6%, greater than or equal to 7%, greater than or equal to 8%, greater than or equal to 9%, greater than or equal to 10%, greater than or equal to 13%, or greater than or equal to 15% as measured by X-ray diffractive spectroscopy. In some embodiments, the polymer has a crystallinity of less than or equal to 40%, less than or equal to 30%, less than or equal to 20%, less than or equal to 18%, less than or equal to 16%, less than or equal to 15%, less than or equal to 14%, less than or equal to 13%, less than or equal to 12%, less than or equal to 11%, less than or equal to 10%, or less than or equal to 9% as measured by X-ray diffractive spectroscopy. Crystallinity and presence thereof of the polymer and the microparticle can be measured by differential scanning calorimetry and/or X-ray diffractive spectroscopy.

Crystallinity of the polymer is useful for providing larger microparticles, such as greater than 5 μm in diameter. For example, the microparticle can have a diameter of about 5 μm to about 500 μm, such as about 5 μm to about 450 μm, about 10 μm to about 400 μm, about 15 μm to about 350 μm, about 20 μm to about 300 μm, about 25 μm to about 250 μm, about 50 μm to about 200 μm, or about 75 μm to about 150 μm. In some embodiments, the microparticle has a diameter of no greater than 500 μm, no greater than 400 μm, no greater than 300 μm, no greater than 200 μm, no greater than 100 μm, no greater than 50 μm, no greater than 25 μm, no greater than 20 μm, no greater than 15 μm, no greater than 10 μm, or no greater than 5 μm. In some embodiments, the microparticle has a diameter of at least 5 μm, at least 10 μm, at least 15 μm, at least 20 μm, at least 25 μm, at least 50 μm, at least 100 μm, at least 200 μm, at least 300 μm at least 400 μm, or at least 500 μm.

The microparticles may be characterized by a variety of methods. For example, microscopy (e.g., transmission electron microscopy or scanning electron microscopy) may be used to examine the morphology and size distribution of the microparticles. Dynamic light scattering or potentiometry (e.g., potentiometric titrations) may be used to measure zeta potentials. Dynamic light scattering may also be utilized to determine particle sizes up to a certain size. Instruments such as the Zetasizer Nano ZS (Malvern Instruments Ltd, Malvern, Worcestershire, UK) may also be used to measure multiple characteristics of the microparticle, such as particle size, polydispersity index, and zeta potential.

The microparticle can have advantageous properties that make it useful for drug delivery. Outside of the size of the microparticle, which can be useful for local and sustained drug release applications, the microparticle can have antioxidant properties by being able to scavenge ROS at, e.g., a site of administration. The microparticle can also be degraded in a manner that is responsive to ROS. The impact of this latter property can be seen in embodiments that include a biologically active agent. For example, as ROS can be significantly elevated in inflamed tissues, the biologically responsive activity of the polymer can allow for the specific or enhanced release of the biologically active agent in these environments. This can facilitate a more targeted and faster release of the agent at the site of administration.

### A. Polymers

The polymer can include recurring units of formula (I)

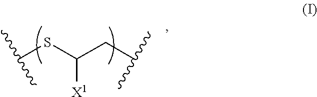

wherein X¹ is H or CH₃. The polymer can include at least one recurring unit of X¹ is H and at least one recurring unit of X¹ is CH₃. The polymer can include a plurality of recurring units of X¹ is H and a plurality of recurring units of X¹ is CH₃.

The polymer can include varying amounts of recurring units where X¹ is CH₃. For example, the polymer can include recurring units of X¹ is CH₃ at about 1 mol % to about 70 mol % of the polymer, such as about 1 mol % to about 60 mol % of the polymer, about 10 mol % to about 60 mol % of the polymer, about 15 mol % to about 60 mol % of the polymer, about 15 mol % to about 55 mol % of the polymer, about 20 mol % to about 55 mol % of the polymer, about 20 mol % to about 50 mol % of the polymer, about 25 mol % to about 50 mol % of the polymer, about 35 mol % to about 60 mol % of the polymer, or about 25 mol % to about 55 mol % of the polymer.

In some embodiments, the polymer includes recurring units of X¹ is CH₃ at less than or equal to 70 mol % of the polymer, less than or equal to 65 mol % of the polymer, less than or equal to 60 mol % of the polymer, less than or equal to 55 mol % of the polymer, less than or equal to 50 mol % of the polymer, less than or equal to 45 mol % of the polymer, less than or equal to 40 mol % of the polymer, less than or equal to 35 mol % of the polymer, less than or equal to 30 mol % of the polymer, less than or equal to 25 mol % of the polymer, less than or equal to 20 mol % of the polymer, less than or equal to 15 mol % of the polymer, or less than or equal to 10 mol % of the polymer.

In some embodiments, the polymer includes recurring units of X¹ is CH₃ at greater than or equal to 1 mol % of the polymer, greater than or equal to 5 mol % of the polymer, greater than or equal to 10 mol % of the polymer, greater than or equal to 15 mol % of the polymer, greater than or equal to 20 mol % of the polymer, greater than or equal to 25 mol % of the polymer, greater than or equal to 30 mol % of the polymer, greater than or equal to 35 mol % of the polymer, greater than or equal to 40 mol % of the polymer, greater than or equal to 45 mol % of the polymer, greater than or equal to 50 mol % of the polymer, greater than or equal to 55 mol % of the polymer, or greater than or equal to 60 mol % of the polymer.

The polymer can include varying amounts of recurring units where X¹ is H. For example, the polymer can include recurring units of X¹ is H at about 30 mol % to about 60 mol % of the polymer, such as about 30 mol % to about 55 mol % of the polymer, about 30 mol % to about 50 mol % of the polymer, about 35 mol % to about 50 mol % of the polymer, about 40 mol % to about 55 mol % of the polymer, or about 35 mol % to about 45 mol % of the polymer. In some embodiments, the polymer includes recurring units of X¹ is H at less than or equal to 60 mol % of the polymer, less than or equal to 55 mol % of the polymer, less than or equal to 50 mol % of the polymer, less than or equal to 45 mol % of the polymer, less than or equal to 40 mol % of the polymer, or less than or equal to 35 mol % of the polymer. In some embodiments, the polymer includes recurring units of X¹ is H at greater than or equal to 30 mol % of the polymer, greater than or equal to 35 mol % of the polymer, greater than or equal to 40 mol % of the polymer, greater than or equal to 45 mol % of the polymer, greater than or equal to 50 mol % of the polymer, or greater than or equal to 55 mol % of the polymer. In some embodiments, the polymer includes recurring units of X¹ is H at greater than 30 mol % of the polymer, greater than 31 mol % of the polymer, greater than 32 mol % of the polymer, greater than 33 mol % of the polymer, greater than 34 mol % of the polymer, greater than 35 mol % of the polymer, greater than 36 mol % of the polymer, greater than 37 mol % of the polymer, greater than 38 mol % of the polymer, or greater than 39 mol % of the polymer.

In some embodiments, the polymer includes recurring units of X¹ is CH₃ at about 45 mol % to about 55 mol % of the polymer, and recurring units of X¹ is H at about 45 mol % to about 55 mol % of the polymer. In some embodiments, the polymer includes recurring units of X¹ is CH₃ at about 30 mol % to about 60 mol % of the polymer, and recurring units of X¹ is Hat about 35 mol % to about 65 mol % of the polymer. In some embodiments, the polymer includes recurring units of X¹ is CH₃ at about 20 mol % to about 60 mol % of the polymer, and recurring units of X¹ is Hat about 40 mol % to about 60 mol % of the polymer.

Recurring units of X¹ is CH₃ and recurring units of X¹ is H can be included at varying molar ratios. For example, recurring units of X¹ is CH₃ and recurring units of X¹ is H can be included in the polymer at a molar ratio of about 70:30 to about 40:60 (X¹ is CH₃: X¹ is H), such as about 65:35 to about 45:55 (X¹ is CH₃: X¹ is H) or about 60:40 to about 50:50 (X¹ is CH₃: X¹ is H). In some embodiments, the recurring units of X¹ is CH₃ and recurring units of X¹ is H are included in the polymer at a molar ratio of about 50:50, about 55:45, or about 45:55 (X¹ is CH₃: X¹ is H).

The recurring units of formula (I) can be repeated a number of times. For example, the recurring unit of formula (I) can be repeated 30 to 3,000 times, such as 50 to 2,500 times, 75 to 2,000 times, 30 to 800 times, 50 to 500 times, or 100 to 400 times. In some embodiments, the recurring unit of formula (I) is repeated less than 3,000 times, less than 2,00 times, less than 1,000 times, less than 500 times, or less than 300 times. In some embodiments, the recurring unit of formula (I) is repeated greater than 30 times, greater than 50 times, greater than 75 times, greater than 100 times, or greater than 200 times. The number of repeats of the recurring unit can also be expressed as a subscript “n” associated with the recurring unit as typically done in the art with polymers. For example, formula (I) can be denoted as follows

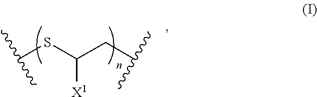

wherein n can be as described above, e.g., 30 to 3,000.

The recurring unit of X¹ is H and the recurring unit of X¹ is CH₃ can also each individually be repeated a number of times. For example, the recurring unit of X¹ is CH₃ can be repeated 25 to 2,000 times, such as 50 to 1,500 times, 75 to 1,000 times, 100 to 800 times, 50 to 500 times, or 100 to 250 times. In some embodiments, the recurring unit of X¹ is CH₃ is repeated less than 2,000 times, less than 1,500 times, less than 1,000 times, less than 500 times, or less than 300 times. In some embodiments, the recurring unit of X¹ is CH₃ is repeated greater than 25 times, greater than 50 times, greater than 75 times, greater than 100 times, or greater than 200 times. In some embodiments, the recurring unit of X¹ is CH₃ is repeated 110 to 220 times.

In addition, the recurring unit of X¹ is H can be repeated 5 to 1,000 times, such as 5 to 800 times, 10 to 900 times, 5 to 500 times, 10 to 300 times, or 5 to 200 times. In some embodiments, the recurring unit of X¹ is H is repeated less than 1,000 times, less than 800 times, less than 600 times, less than 400 times, or less than 200 times. In some embodiments, the recurring unit of X¹ is His repeated greater than 5 times, greater than 10 times, greater than 25 times, greater than 50 times, or greater than 100 times. In some embodiments, the recurring unit of X¹ is His repeated 5 to 110 times.

The polymer can also be described as a random copolymer that can include recurring units of formula (II)

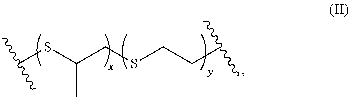

wherein x is about 1 mol % to about 70 mol % of the polymer; and y is greater than or equal to 30 mol % of the polymer. Because formula (II) refers to a random copolymer, recurring units of x and y can be included in the polymer randomly, and not as blocks of recurring units of x and y. Accordingly, the polymer can be a random copolymer.

In some embodiments, x is about 1 mol % to about 70 mol % of the polymer. In some embodiments, x is about 10 mol % to about 60 mol % of the polymer, about 15 mol % to about 60 mol % of the polymer, about 15 mol % to about 55 mol % of the polymer, about 20 mol % to about 55 mol % of the polymer, about 20 mol % to about 50 mol % of the polymer, about 25 mol % to about 50 mol % of the polymer, or about 25 mol % to about 55 mol % of the polymer. In some embodiments, x is no greater than 60 mol % of the polymer, no greater than 55 mol % of the polymer, no greater than 50 mol % of the polymer, no greater than 45 mol % of the polymer, no greater than 40 mol % of the polymer, no greater than 35 mol % of the polymer, no greater than 30 mol % of the polymer, no greater than 25 mol % of the polymer, no greater than 20 mol % of the polymer, no greater than 15 mol % of the polymer, or no greater than 10 mol % of the polymer. In some embodiments, x is at least 10 mol % of the polymer, at least 15 mol % of the polymer, at least 20 mol % of the polymer, at least 25 mol % of the polymer, at least 30 mol % of the polymer, at least 35 mol % of the polymer, at least 40 mol % of the polymer, at least 45 mol % of the polymer, at least 50 mol % of the polymer, at least 55 mol % of the polymer, or at least 60 mol % of the polymer.

In some embodiments, y is about 30 mol % to about 60 mol % of the polymer. In some embodiments, y is about 30 mol % to about 55 mol % of the polymer, about 30 mol % to about 50 mol % of the polymer, about 35 mol % to about 50 mol % of the polymer, about 40 mol % to about 55 mol % of the polymer, or about 35 mol % to about 45 mol % of the polymer. In some embodiments, y is no greater than 60 mol % of the polymer, no greater than 55 mol % of the polymer, no greater than 50 mol % of the polymer, no greater than 45 mol % of the polymer, no greater than 40 mol % of the polymer, or no greater than 35 mol % of the polymer. In some embodiments, y is at least 30 mol % of the polymer, at least 35 mol % of the polymer, at least 40 mol % of the polymer, at least 45 mol % of the polymer, at least 50 mol % of the polymer, at least 55 mol % of the polymer, or at least 60 mol % of the polymer.

In some embodiments the polymer further includes recurring units of formula (III):

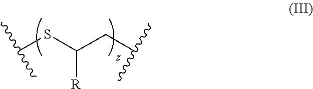

wherein R is —C₁₋₂alkylene-R¹¹, —C₁₋₂alkylene-OC₁₋₂alkylene-R¹¹, or phenyl; R¹¹ is G¹, C₁₋₄alkyl, C₃₋₄cycloalkyl, cyano, —OH, —OC₁₋₄alkyl, —OSi(C₁₋₄alkyl) 3, —OC₁₋₂haloalkyl, —OC₃₋₄cycloalkyl, —C(O) C₁₋₄alkyl, or —CO₂C₁₋₄alkyl; and G¹ is phenyl, wherein G¹ is optionally substituted with 1 or 2 substituents, each independently halogen, cyano, C₁₋₄alkyl, C₁₋₂fluoroalkyl, —OC₁₋₂alkyl, or —OC₁₋₂fluoroalkyl.

In some embodiments, R is-C₁₋₂alkylene-R¹¹, —C₁₋₂alkylene-OC₁₋₂alkylene-R¹¹, or phenyl; R¹¹ is G¹, C₁₋₄alkyl, C₃₋₄cycloalkyl, —OH, —OC₁₋₄alkyl, —OSi(C₁₋₄alkyl) 3, —OC₁₋₂haloalkyl, —OC₃₋₄cycloalkyl, or —CO₂C₁₋₄alkyl; and G¹ is phenyl, wherein G¹ is optionally substituted with 1 or 2 substituents, each independently halogen, C₁₋₄alkyl, or —OC₁₋₂alkyl.

In some embodiments, R is-C₁₋₂alkylene-R¹¹ or phenyl; R¹¹ is G¹, C₁₋₄alkyl, or —OH; and G¹ is phenyl, wherein G¹ is optionally substituted with 1 or 2 substituents, each independently halogen, C₁₋₄alkyl, or —OC₁₋₂alkyl.

In some embodiments, R is-C₁₋₂alkylene-R¹¹ or phenyl; R¹¹ is G¹ or —OH; and G¹ is phenyl, wherein G¹ is optionally substituted with 1 or 2 substituents, each independently halogen, or —OC₁₋₂alkyl.

In some embodiments, R is selected from the group consisting of:

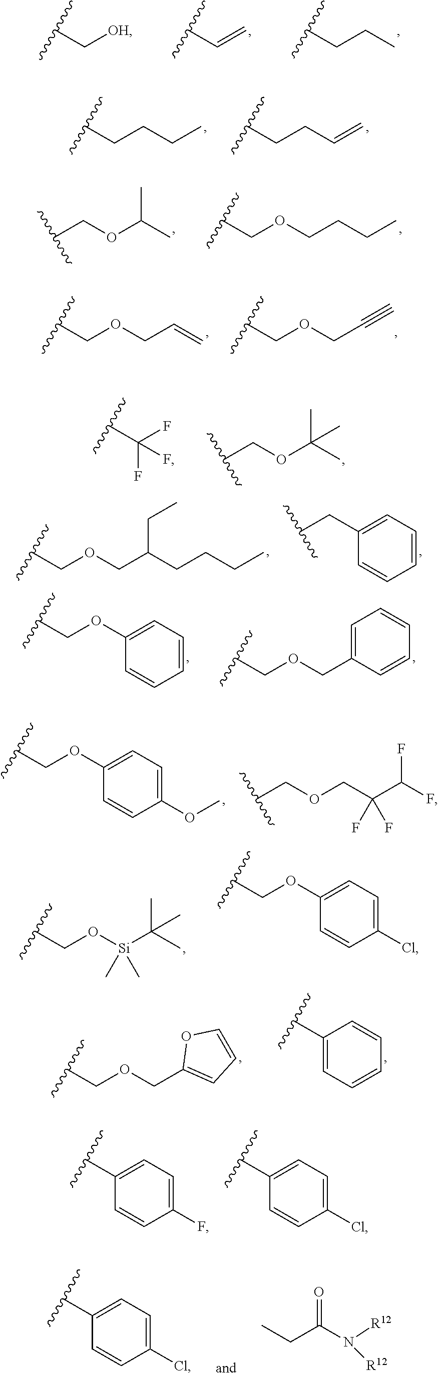

(wherein R¹² is H or alkyl).

In some embodiments, R is selected from the group consisting of:

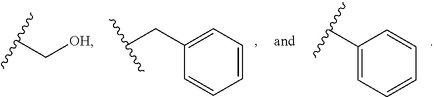

The polymer can include varying amounts of recurring units of formula (III). For example, z can be about 0.1 mol % to about 60 mol % of the polymer, such as about 1 mol % to about 60 mol % of the polymer, about 1 mol % to about 50 mol % of the polymer, about 1 mol % to about 40 mol % of the polymer, about 0.5 mol % to about 25 mol % of the polymer, about 1 mol % to about 25 mol % of the polymer, about 1 mol % to about 20 mol % of the polymer, about 1 mol % to about 15 mol % of the polymer, about 0.1 mol % to about 10 mol % of the polymer, about 1 mol % to about 10 mol % of the polymer, or about 1 mol % to about 5 mol % of the polymer. In some embodiments, z is greater than or equal to 0.1 mol % of the polymer, greater than or equal to 0.5 mol % of the polymer, greater than or equal to 1 mol % of the polymer, greater than or equal to 5 mol % of the polymer, greater than or equal to 10 mol % of the polymer, greater than or equal to 15 mol % of the polymer, greater than or equal to 20 mol % of the polymer, or greater than or equal to 25 mol % of the polymer. In some embodiments, z is less than or equal to 60 mol % of the polymer, less than or equal to 50 mol % of the polymer, less than or equal to 40 mol % of the polymer, less than or equal to 30 mol % of the polymer, less than or equal to 20 mol % of the polymer, or less than or equal to 10 mol % of the polymer.

Recurring units of formula (III), wherein R is defined as described above, may be prepared as shown in the general reaction scheme:

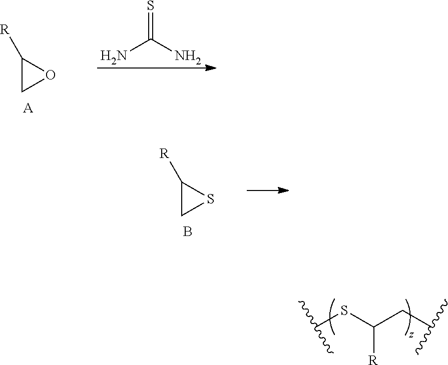

As shown in the general scheme above, R-substituted oxiranes of formula A may be reacted with thiourea under suitable conditions known to those of ordinary skill in the art to form the corresponding R-substituted thiiranes of formula B. Thiiranes of formula B may subsequently be polymerized under suitable ring-opening polymerization conditions known to those of ordinary skill in the art, and which are disclosed herein, to form recurring units of formula (III), wherein z is defined as described above in formula (III). R-substituted oxiranes of formula A may also be bought commercially.

The polymer can have a varying number average molecular weight. For example, the polymer can have a number average molecular weight of about 1,000 Daltons (Da) to about 200,000 Da, such as about 2,000 Da to about 150,000 Da, about 5,000 Da to about 100,000 Da, about 8,000 Da to about 50,000 Da, or about 10,000 Da to about 20,000 Da. In some embodiments, the polymer has a number average molecular weight of greater than 1,000 Da, greater than 2,000 Da, greater than 5,000 Da, greater than 10,000 Da, greater than 15,000 Da, greater than 50,000 Da, or greater than 100,000 Da. In some embodiments, the polymer has a number average molecular weight of less than 200,000 Da, less than 150,000 Da, less than 100,000 Da, less than 50,000 Da, less than 40,000 Da, less than 30,000 Da, or less than 20,000 Da. In some embodiments, the polymer has a number average molecular weight of about 11,500 Da to about 18,000 Da.

Molecular weight of the polymer can be measured by techniques known within the art, such as size exclusion chromatography (SEC), SEC combined with multi-angle light scattering, gel permeation chromatography, rheometry, and the like.

i. Methods of Making Polymers

Further provided herein are methods of making the polymer. The method may include adding a first sulfide monomer and a second sulfide monomer to a reaction mixture via a syringe pump. The sulfide monomers can be monomers that provide recurring units of formula (I), formula (II), formula (III), or a combination thereof. In some embodiments, the first sulfide monomer is propylene sulfide (PS). In some embodiments, the second sulfide monomer is ethylene sulfide (ES). In some embodiments, more than two sulfide monomers can be added, such as a third sulfide monomer, a fourth sulfide monomer, and so on. Monomers in addition to the first sulfide monomer and the second sulfide monomer can be a monomer that can provide recurring units of formula (III). The monomers may be added to the reaction mixture in the form of a monomer solution.

The monomers can be added in varying ratios to provide the disclosed polymers. For example, the first sulfide monomer and the second sulfide monomer may be added at a molar ratio of about 95:5 (first monomer: second monomer), about 90:10, about 80:20; about 70:30, about 60:40, about 55:45, about 50:50, about 45:55, about 40:60, or about 35:60. The syringe pump may allow for a slow addition of each monomer to the reaction mixture. The addition of the monomers or the solutions thereof may be completed over a period of hours, such as about 1 hour, 2 hours, 3 hours, or 4 hours. In some embodiments, the sulfide monomers are added to the reaction mixture via the syringe pump at less than or equal to 15 μl/minute (min), less than or equal to 14 μl/min, less than or equal to 13 μl/min, less than or equal to 12 μl/min, less than or equal to 11 μl/min, or less than or equal to 10 μl/min. In some embodiments, the sulfide monomers are added to the reaction mixture via the syringe pump at greater than or equal to 1 μl/min, greater than or equal to 2 μl/min, greater than or equal to 3 μl/min, greater than or equal to 4 μl/min, or greater than or equal to 5 μl/min. In some embodiments, the sulfide monomers are added to the reaction mixture via the syringe pump at about 1 μl/min to about 15 μl/min, such as about 2 μl/min to about 14 μl/min, about 3 μl/min to about 13 μl/min, about 4 μl/min to about 12 μl/min, about 3 μl/min to about 15 μl/min, or about 5 μl/min to about 12 μl/min. Further, regarding the addition of the monomers or solutions thereof, said monomers or solutions thereof may be added sequentially or continuously.

In the reaction mixture, the monomers can be polymerized via an anionic ring opening polymerization. The polymer provided can be a random copolymer with crystalline properties that provide useful advantages in e.g., drug delivery applications. For example, the polymer provided can be a random copolymer having a crystallinity of about 1% to about 40% as measured by X-ray diffractive spectroscopy, such as about 3% to about 38%, about 5% to about 35%, about 1% to about 20%, about 5% to about 20%, about 5% to about 15%, or about 15% to about 40% as measured by X-ray diffractive spectroscopy.

Alternatively, the polymer can be made by adding the monomers in multiple additions, e.g. by hand, burette, etc., spaced apart from each other. For example, in some embodiments, each sulfide monomer can be added in equal volumes spaced by a time interval, such as 10 minutes, 15 minutes, or 20 minutes apart from each addition. In some embodiments, the sulfide monomers are added in 5, 6, 7, 8, 9, 10, or more individually equal volumes separated by a time interval as described above.

The polymer provided can be a polymer including recurring units of formula (I), (II), (III), or a combination thereof. In some embodiments, the polymer provided is a polymer including recurring units of formula (I).

### B. Biologically Active Agents

The microparticle may further include a biologically active agent. Examples of biologically active agents include, but are not limited to, organic catalyst, antibiotic, antioxidant, anti-ROS agent, anti-inflammatory, protein, glycoprotein, peptide, polyamino acid, antibody, epitopes of antibodies, nucleic acid, steroidal molecule, antiviral, antirejection agent, immunosuppressant, cytokine, carbohydrate, pharmaceutical, cell, virus, single chain fragment, siRNA, miRNA against the p53/MAP kinase pathway, virus vector, prion, anti-proliferative agent, anti-migratory agent, biologically active polymer, and combinations thereof. In some embodiments, the biologically active agent includes at least one of an enzyme, an organic catalyst, an antibiotic, an antioxidant, an anti-ROS agent, an anti-inflammatory, a protein, a glycoprotein, a peptide, a polyamino acid, a nucleic acid, a steroidal molecule, an antiviral, an antirejection agent, an immunosuppressant, a carbohydrate, a pharmaceutical, a cell, a virus, a virus vector, an anti-proliferative agent, an anti-migratory agent, a biologically active polymer, and a combination thereof. In some embodiments, the biologically active agent includes a cytokine, an antioxidant, an anti-ROS agent, an anti-inflammatory, a nucleic acid, or a combination thereof. In some embodiments, the biologically active agent includes a cytokine, a siRNA, an antibody, or a combination thereof. In some embodiments, the biologically active agent includes a cytokine, a RNA, an antibody, or a combination thereof. In some embodiments, the biologically active agent includes erythropoietin, siRNA, or a combination thereof. In some embodiments, the biologically active agent includes erythropoietin or siRNA. In some embodiments, the biologically active agent includes erythropoietin.

The biologically active agent can be included in the microparticle at varying amounts. For example, the microparticle can include the biologically active agent at about 0.05% to about 2% by weight of the microparticle, such as about 0.1% to about 15% by weight of the microparticle, about 1% to about 2% by weight of the microparticle, or about 0.05% to about 0.5% by weight of the microparticle. In some embodiments, the microparticle includes the biologically active agent at greater than 0.05% by weight of the microparticle, greater than 0.1% by weight of the microparticle, or greater than 0.5% by weight of the microparticle. In some embodiments, the microparticle includes the biologically active agent at less than 2% by weight of the microparticle, less than 1.5% by weight of the microparticle, or less than 1% by weight of the microparticle. In some embodiments, the microparticle includes the biologically active agent at about 0.05% to about 1.1% by weight of the microparticle.

### C. Methods of Making Microparticles

Also provided herein are methods of making the microparticle. Microparticles can be made through polymer emulsion techniques known within the art. For example, the microparticles can be made via emulsion techniques used to provide poly-(lactic-co-glycolic acid) (PLGA)-based particles. Accordingly, the disclosed microparticles can be made through emulsion techniques including, but not limited to, oil-in-water (O/W) emulsion, water-in-oil-in-water (W/OW) emulsion, and solid-in-oil-in-water (S/O/W) emulsion.

The method can also include a novel step to improve drug loading of the biologically active agent within the microparticle. The method may include adding a nanoparticle comprising a dextran or other polymer and the biologically active agent to a first solvent to provide a mixture, wherein the first solvent comprises a polymer including recurring units of formula (I) as defined above. The first solvent can be any suitable solvent that can dissolve the polymer. An example solvent includes, but is not limited to, methylene chloride (DCM).

The method can also include adding a second solvent to the mixture and emulsifying the mixture to provide a microparticle that includes the polymer and the nanoparticle. The nanoparticle can be dispersed in the microparticle, where the biologically active agent is present in the nanoparticle. The second solvent can be any suitable solvent having miscibility with the first solvent and that is a poor solvent for dissolving the polymer. An example second solvent includes, but is not limited to, polyvinyl alcohol (PVA). The first solvent can be considered an oil phase of an emulsion. The second solvent can be considered the water phase of an emulsion. Accordingly, the second solvent can be more polar compared to the first solvent. The method can further include preparing the nanoparticle prior to adding it to the first solvent. The method of making the microparticle using dextran can be referred to as a solid-in-oil-in-water (S/O/W) emulsion.

Dextran or other polymers can be used to provide higher loading of the biologically active agent within the microparticle, as well as may enhance the stability of the biologically active agent during the emulsion process and within the microparticle. Dextran may be used to interact with the biologically active agent, e.g., via electrostatic interactions. For example, dextran may be cationic dextran (e.g., have a net positive charge) and may interact with a negative charge present on the biologically active agent. The amount of cationic charge on dextran can be modulated to increase loading of various agents.

The dextran can be included in the microparticle in varying amounts. For example, the microparticle can include the dextran at about 1% to about 20% by weight of the microparticle, such as about 1% to about 15% by weight of the microparticle, about 2% to about 15% by weight of the microparticle, about 1% to about 10% by weight of the microparticle, about 3% to about 10% by weight of the microparticle, about 4% to about 10% by weight of the microparticle, or about 1% to about 5% by weight of the microparticle. In some embodiments, the microparticle includes dextran at no greater than 20% by weight of the microparticle, no greater than 15% by weight of the microparticle, no greater than 10% by weight of the microparticle, no greater than 7% by weight of the microparticle, no greater than 5% by weight of the microparticle, no greater than 3% by weight of the microparticle, or no greater than 1% by weight of the microparticle. In some embodiments, the microparticle includes dextran at no less than 1% by weight of the microparticle, no less than 3% by weight of the microparticle, no less than 1% by weight of the microparticle, no less than 3% by weight of the microparticle, no less than 5% by weight of the microparticle, no less than 7% by weight of the microparticle, no less than 10% by weight of the microparticle, or no less than 15% by weight microparticle.

### 3. METHODS OF TREATING INFLAMMATORY DISEASES

Also provided herein are methods of treating an inflammatory disease. The inflammatory disease may be a disease associated with either acute or chronic inflammation caused by oxidative stress. Example inflammatory diseases include, but are not limited to, glaucoma, arthritis (e.g., osteoarthritis, rheumatoid arthritis, etc.), pancreatitis, fibrotic lung disease (e.g., pustular fibrosis, COPD, and idiopathic pulmonary fibrosis), atherosclerosis, ischemia-reperfusion injury, stroke, autoimmune disease, autism, macular degeneration, traumatic brain injury, multiple sclerosis, cancer, diabetes, and muscular dystrophy. In some embodiments, the inflammatory disease is a chronic inflammatory disease. In some embodiments, the inflammatory disease is glaucoma, osteoarthritis, rheumatoid arthritis, psoriatic arthritis, pancreatitis, multiple sclerosis, cancer, diabetes, traumatic brain injury, fibrotic lung disease, atherosclerosis, ischemia-reperfusion injury, stroke, autoimmune disease, autism, macular degeneration, indirect traumatic optic neuropathy, or muscular dystrophy. In some embodiments, the inflammatory disease is glaucoma, osteoarthritis, pancreatitis, fibrotic lung disease, atherosclerosis, ischemia-reperfusion injury, stroke, autoimmune disease, autism, macular degeneration, or muscular dystrophy. In some embodiments, the inflammatory disease is glaucoma, macular degeneration, indirect traumatic optic neuropathy, or osteoarthritis. In some embodiments, the inflammatory disease is glaucoma or osteoarthritis. In some embodiments, the inflammatory disease is glaucoma, indirect traumatic optic neuropathy, or macular degeneration. In some embodiments, the inflammatory disease is glaucoma.

The method can include administering to a subject (in need thereof) an effective amount of the microparticle as disclosed herein. In some embodiments, the subject is human. In addition, the microparticle can be administered to the subject optionally in combination with a pharmaceutically acceptable excipient. Embodiments that administer the microparticle in combination with a pharmaceutically acceptable excipient can also be referred to as a pharmaceutical composition. Examples of pharmaceutically acceptable excipients include, but are not limited to, buffering agents (e.g., phosphate buffered saline, artificial cerebrospinal fluid (aCSF), etc.), carbohydrates (e.g., glucose, trehalose, starch, etc.) solubilizers, solvents, antimicrobial preservatives, antioxidants, suspension agents, penetration/absorption enhancers (e.g., DMSO, ethanol, pyrrolidones, and/or ionic liquids), polyvinyl alcohol, pluronics, carboxymethylcellulose, or a combination thereof.

In some embodiments, the pharmaceutically acceptable excipient includes saline, albumin, dimethyl sulfoxide, trehalose, sucrose, polyethylene glycol, polyvinyl alcohol, pluronics, carboxymethylcellulose, an absorption enhancer, or a combination thereof. In some embodiments, the pharmaceutically acceptable excipient includes saline, albumin, dimethyl sulfoxide, trehalose, sucrose, polyethylene glycol (PEG), an absorption enhancer, or a combination thereof. In some embodiments, the microparticle is administered in combination with a pharmaceutically acceptable excipient.

The microparticle or pharmaceutical composition thereof can be administered prophylactically or therapeutically. In prophylactic administration, the microparticle or pharmaceutical composition thereof may be administered in an effective amount to induce a prophylactic response. In therapeutic applications, the microparticle or pharmaceutical composition thereof may be administered to a subject in need thereof in an effective amount to elicit a therapeutic effect. Amounts effective for this use will depend on, e.g., the particular microparticle regimen administered, the manner of administration, the stage and severity of the disease, the general state of health of the patient, and the judgment of the prescribing physician.

The microparticle or pharmaceutical composition thereof may be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, and subjects treated, the particular agents employed, and the specific use for which these agents are employed. The determination of effective dosage levels, that is the dosage levels necessary to achieve the desired result, can be accomplished by one skilled in the art using routine methods, for example, human clinical trials, in vivo studies and in vitro studies.

The microparticle or pharmaceutical composition thereof can be administered via a variety of routes. Example delivery routes include, but are not limited to, parenteral administration, e.g., intradermal, intramuscular, subcutaneous, and intravitreal delivery. In some embodiments, the microparticle of pharmaceutical composition thereof is administered intravitreally, subcutaneously, intradermally, intramuscularly, or intraperitoneally. In some embodiments, the microparticle or pharmaceutical composition thereof is administered intravitreally.

Dosage amount and interval may be adjusted individually to provide plasma levels of the biologically active agent which are sufficient to maintain the modulating effects, or minimal effective concentration (MEC). The MEC will vary for each agent but can be estimated from in vivo and/or in vitro data. Dosages necessary to achieve the MEC will depend on individual characteristics and route of administration. However, assays well known to those in the art can be used to determine plasma concentrations. Dosage intervals can also be determined using MEC value. Microparticles or pharmaceutical compositions thereof can be administered using a regimen which maintains plasma levels above the MEC for 10-90% of the time, such as between 30-90% or between 50-90%. In cases of local administration or selective uptake, the effective local concentration of the biologically active agent may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest can vary with the severity of the symptoms to be treated and the route of administration. Further, the dose, and perhaps dose frequency, can also vary according to the age, body weight, and response of the individual patient. A program comparable to that discussed above may be used in veterinary medicine.

The disclosed microparticles and methods thereof can provide numerous advantages in the treatment of diseases and disorders. For example, the microparticles and methods thereof can decrease ROS levels at a site of administration, even without a biologically active agent present, compared to not receiving the microparticle. As discussed elsewhere, the microparticle can have beneficial antioxidant properties and can be oxidation responsive. ROS can be significantly elevated in inflamed tissues, and the biologically responsive activity of the microparticles can scavenge ROS. In addition, the biologically responsive activity can provide for the specific and/or enhanced biologically active agent released in these environments. This can permit a more targeted and faster release of drug at the diseased location. The microparticles and methods thereof can also improve functional properties of the tissue at the site of administration, such as improved visual function of the eye (e.g., improved retinal ganglion function, bipolar function, and signal transmission to the visual cortex) compared to not receiving the microparticle.

The description of the microparticles, polymers, and biologically active agents above can also be applied to the methods of treating inflammatory diseases disclosed herein.

The present disclosure has multiple aspects, illustrated by the following non-limiting examples.

### 4. EXAMPLES

### Example 1

### Polysulfide Microparticles with Tunable Crystallinity and ROS Reactivity

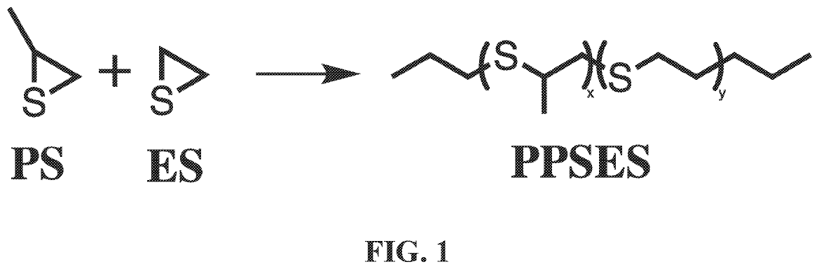

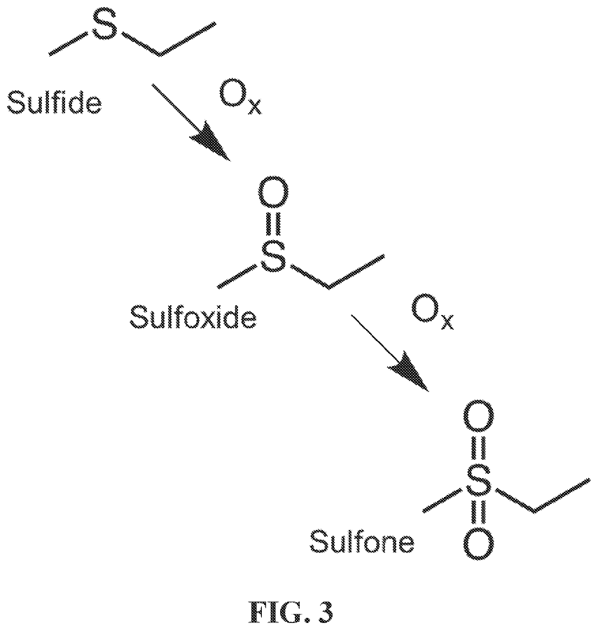

Disclosed herein are novel formulations of polysulfide polymers. These formulations can include drug releasing microparticles that can have tunable size, crystallinity, and ROS reactivity. These are of high interest to the biomedical field as the thioether/polysulfide backbone of polysulfides have potent antioxidant properties and are oxidation responsive. For example, upon oxidation, the polymer switches from hydrophobic to hydrophilic, leading to degradation/drug release in a manner that is responsive to biological reactive oxygen species (ROS) in the local environment (FIG. 3). In this example, the polysulfide included two monomers: amorphous propylene sulfide (PS) and crystalline ethylene sulfide (ES) (FIG. 1). The ratio of these two monomers (PS and ES) may be varied (i.e., PS:ES mol % of 100:0, 90:10, 80:20, 70:30, 60:40, 50:50) to tune the overall polysulfide crystallinity. The monomers were polymerized via an anionic ring-opening polymerization. The use of a syringe pump for slow monomer addition was important as ES polymerizes faster than PS and forms insoluble tapered/gradient polymers if monomer addition is too fast.

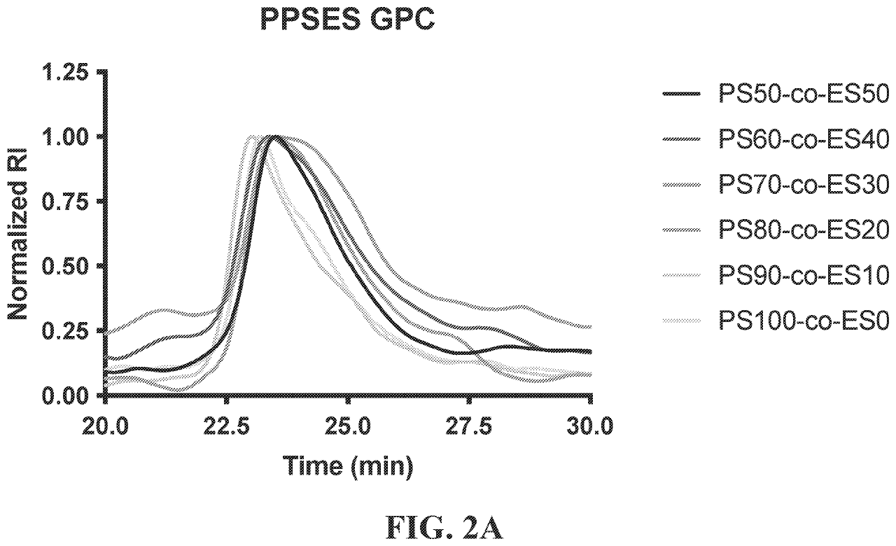

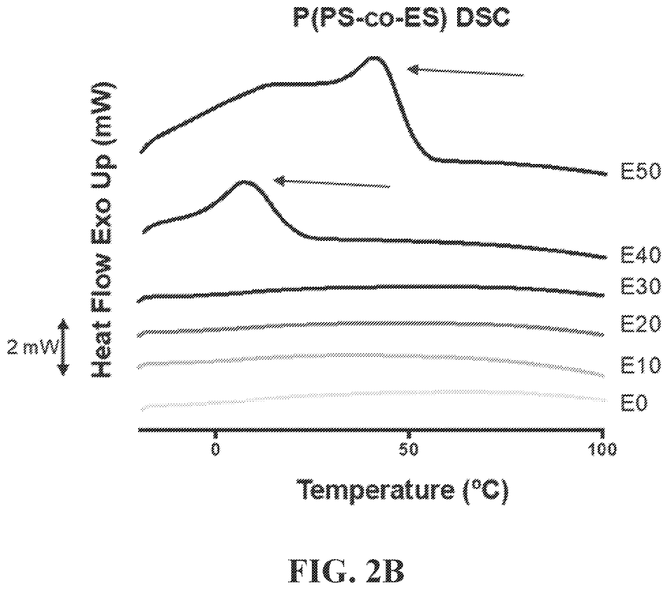

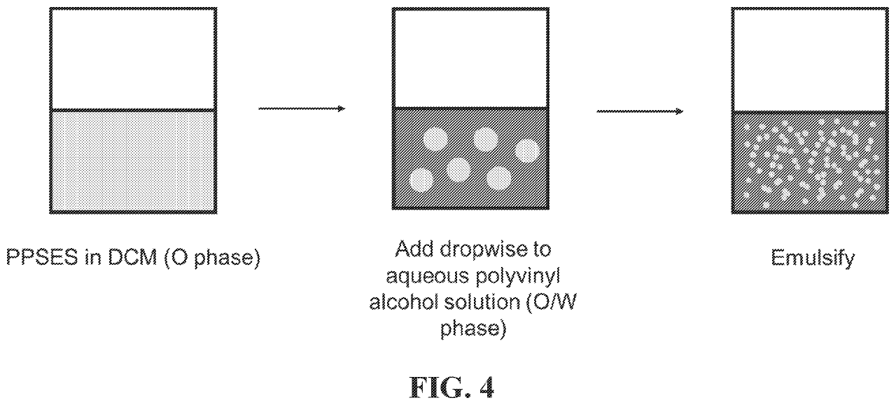

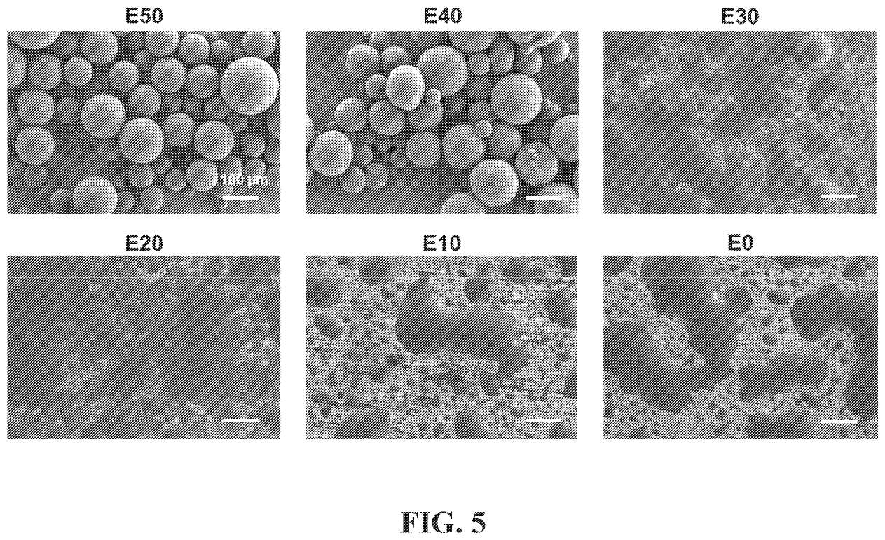

The incorporation of ES into the polymer showed unimodal molecular weight distributions (FIG. 2A). In addition, the incorporation of ES into the polymer above 40 mol % was observed to lend crystallinity to the polymeric PPSES system as measured by differential scanning calorimetry (FIG. 2B). The crystallinity imparted by ES is important to obtaining stable microparticles at sizes above 5 μm (FIG. 5). Unlike prior poly(propylene sulfide) (PPS) (amorphous) polymers, crystalline PPSES (40 or 50 mol % ES) can be formulated into large (50 μm to >280 μm), drug releasing microparticles using a classic bulk oil-in-water (O/W) emulsion technique with poly(vinyl alcohol) as an emulsifier (FIGS. 4-5).

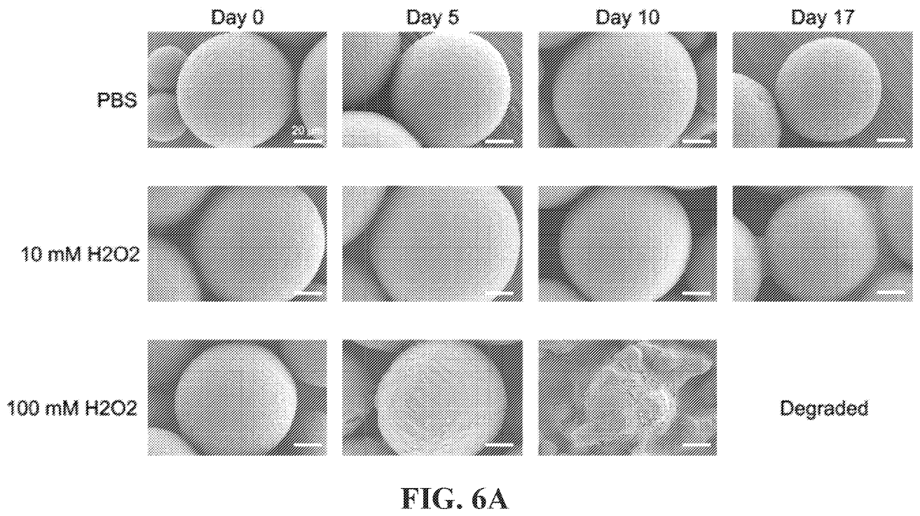

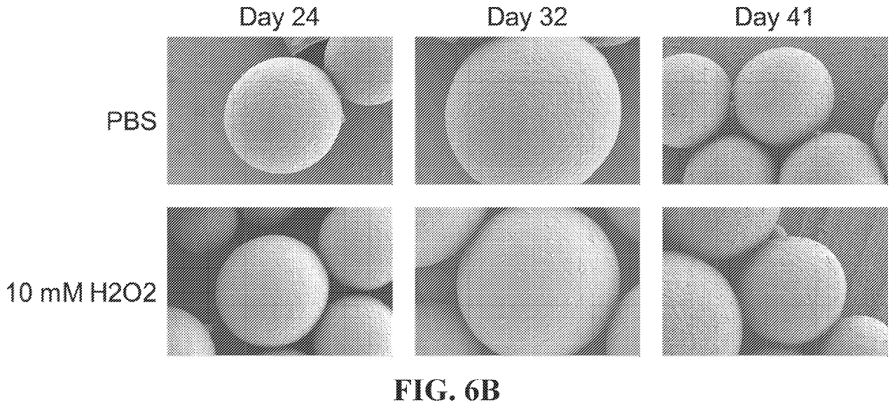

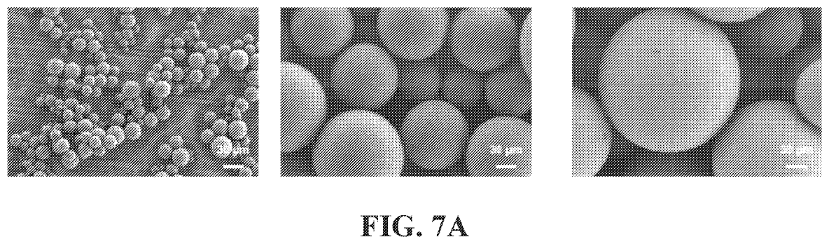

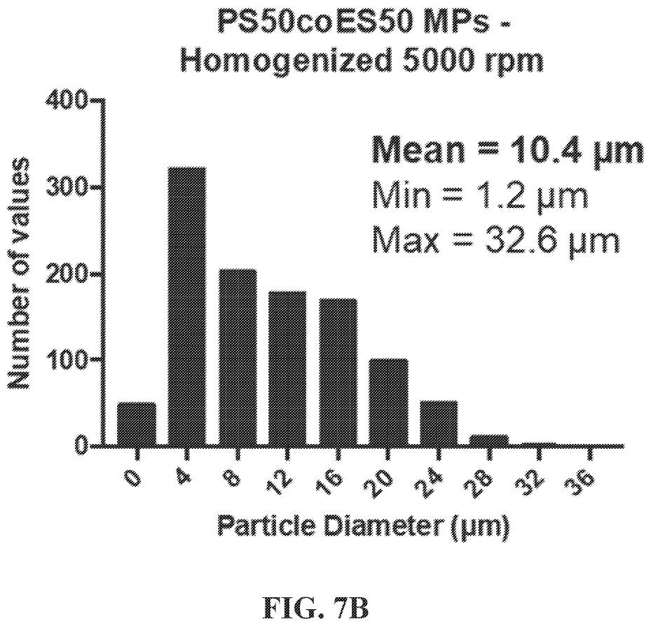

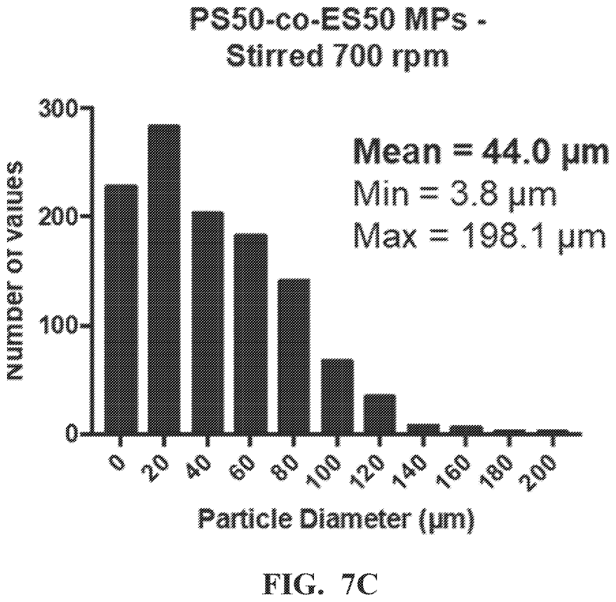

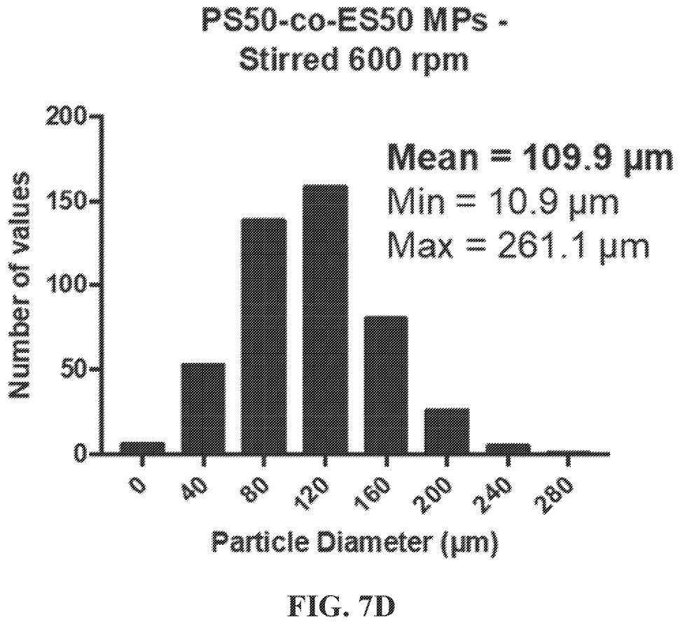

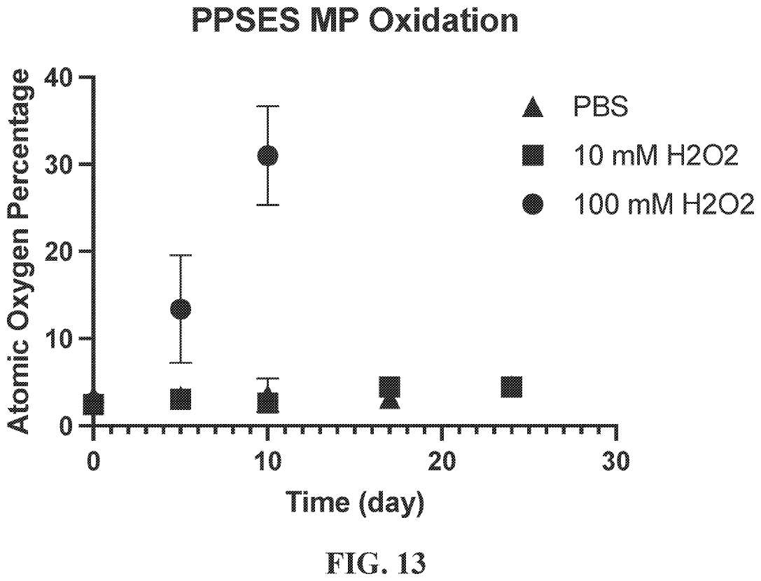

The PPSES microparticle's ROS-responsive degradation was further confirmed in biologically relevant hydrogen peroxide (FIG. 6A, FIG. 6B, and FIG. 13). ROS responsiveness has been found to be dependent on monomer composition, with higher levels of ES resulting in a more rapid degradation. Microparticles of PPSES with 50 mol % ES (E50) can be formulated in broad size distributions by tuning the emulsion parameters such as stir speed or homogenization rate (FIG. 7A, FIG. 7B, FIG. 7C, and FIG. 7D).

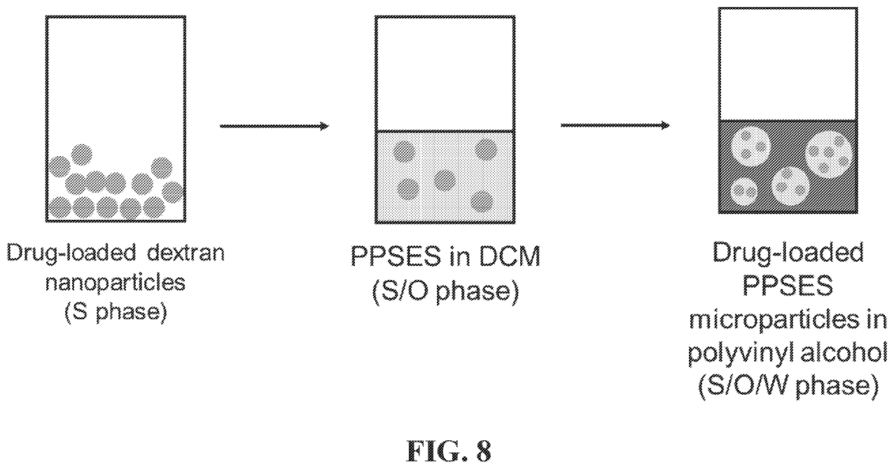

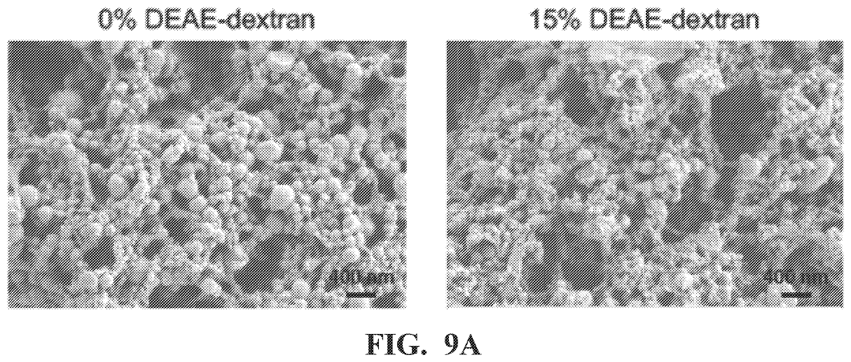

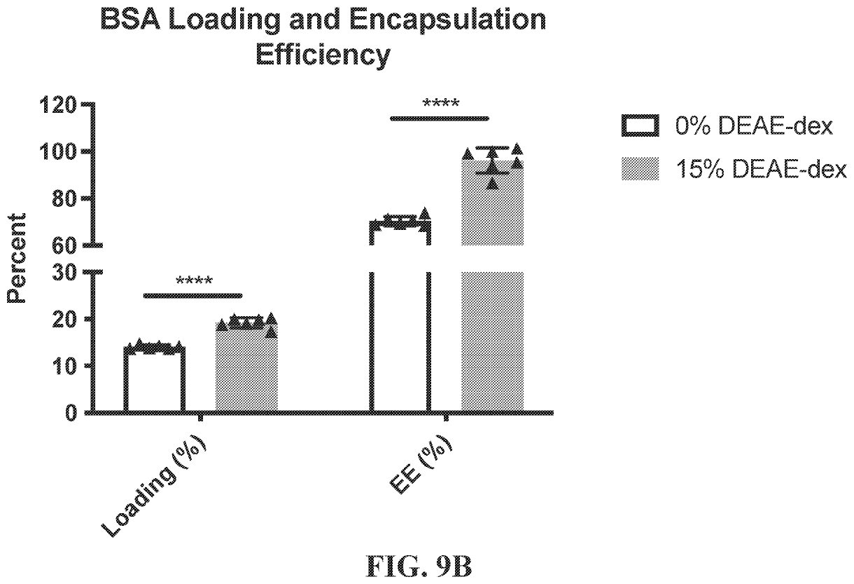

Multiple techniques can be used for loading drugs within PPSES crystalline microparticles for sustained release. Hydrophobic small molecule drugs can be loaded into the polymer oil phase, whereas hydrophilic drugs including biologics can be loaded via a water-in-oil-in-water (W/O/W) or a solid-in-oil-in-water (S/O/W) emulsion (FIG. 8). The model protein drug bovine serum albumin (BSA) was loaded into E50 microparticles using the S/O/W method. First, the protein was formulated into dextran precursor nanoparticles using an aqueous two-phase system technique, then these dextran/BSA solid particles were loaded into the E50 microparticles. Dextran modified with diethylaminoethyl (DEAE) groups was incorporated at various mass ratios to impart cationic character and promote complexation of the negatively charged protein, a novel strategy for improving protein loading (FIG. 9A and FIG. 9B). The mass ratio of cationic dextran:unmodified dextran may be adjusted for other types of anionic cargo, including, but not limited to, siRNA drugs. This is believed to be the first example of doping cationic dextran for S/O/W protein encapsulation to promote better loading of anionic cargoes.

### Example 2

### Antioxidant Microparticles for Delivery of Erythropoietin in a Mouse Glaucoma Model

This example is directed to formulating EPO-loaded MPs using an antioxidant polymer for treatment of a mouse model of glaucoma. Erythropoietin (EPO) is a pleiotropic cytokine which is neuroprotective in part due to its ability to stimulate Nrf2 antioxidant signaling. The administration of AAV-EPO reduces glaucoma progression, however, the protein itself exhibits a short half-life intravitreally (˜3 days). Local injection of EPO-loaded microparticles (MPs) can extend retention in the vitreous and utilizing an antioxidant carrier to synergize with Nrf2 activation improves therapeutic outcomes, unlike classically used polyester formulations.

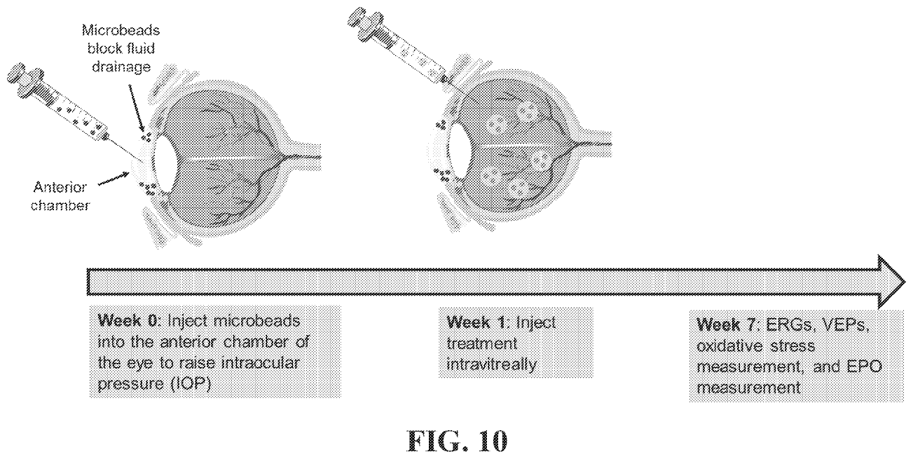

Poly(propylene sulfide-co-ethylene sulfide) (PPSES) was synthesized using ring opening polymerization with an equimolar feed of each monomer. EPO-loaded MPs were formulated by first producing EPO-loaded dextran nanoparticles and loading these into PPSES MPs. The S/O/W technique was used to load EPO. To establish the microbead occlusion model (MOM, FIG. 10) of glaucoma, polystyrene beads were injected into the anterior chamber of C57/B16 mice. Saline-injected mice served as healthy controls. After 1 week, phosphate buffered saline (PBS)-free EPO, blank MPs, or EPO-loaded MPs were injected intravitreally. At 6 weeks post-MP injection, electroretinograms (ERGs) and visual evoked potential (VEP) tests were performed intravitally. Retinal tissue was evaluated ex vivo for EPO and ROS levels using ELISA and Amplex Red dye, respectively.

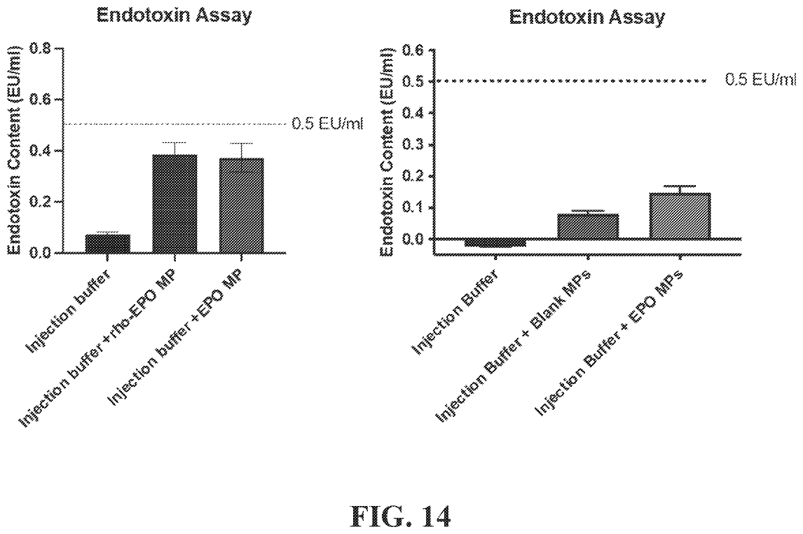

Microparticles can also be administered to mice with low levels of endotoxin (FIG. 14). Briefly, for the endotoxin analysis, microparticles were suspended in injection buffer (PBS+0.25% pluronic F127+0.25% carboxymethylcellulose) at the working concentration for intravitreal injections (5 mg/ml), and endotoxin was quantified using the commercial Pierce Chromogenic Endotoxin Assay Kit (Thermo Fisher).

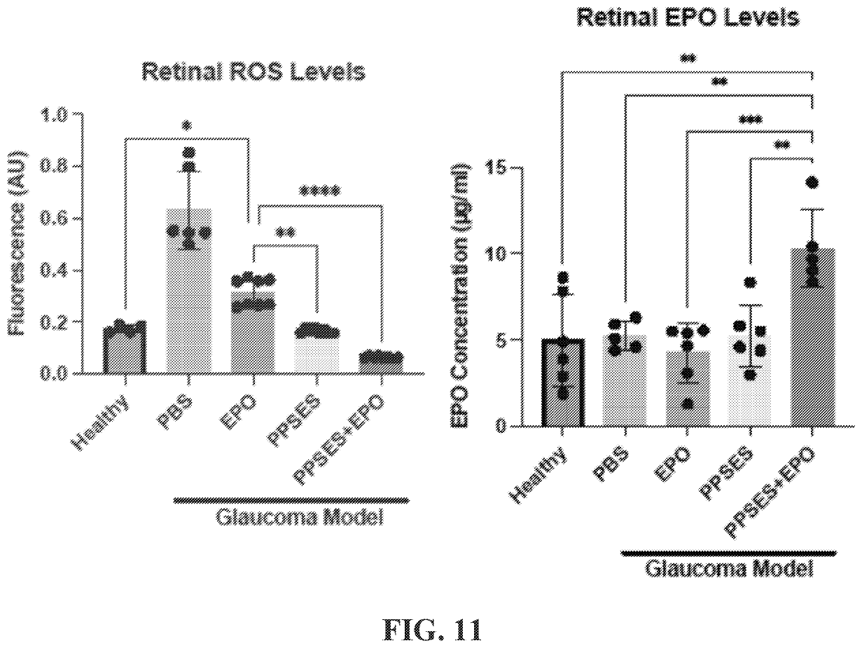

The PPSES polymer yielded uniform and consistent ˜10-100 μm diameter MPs. Injection of PPSES-EPO sustained EPO release out to 6 weeks over EPO alone, which was not different from PBS animals (FIG. 11—left panel). PPSES MPs reduced retinal ROS levels due to ROS scavenging by the backbone thioethers, and this effect was compounded by antioxidant signaling by EPO in the PPSES-EPO groups (FIG. 11—right panel). Both groups outperformed free EPO, demonstrating the benefit of persistence in vivo.

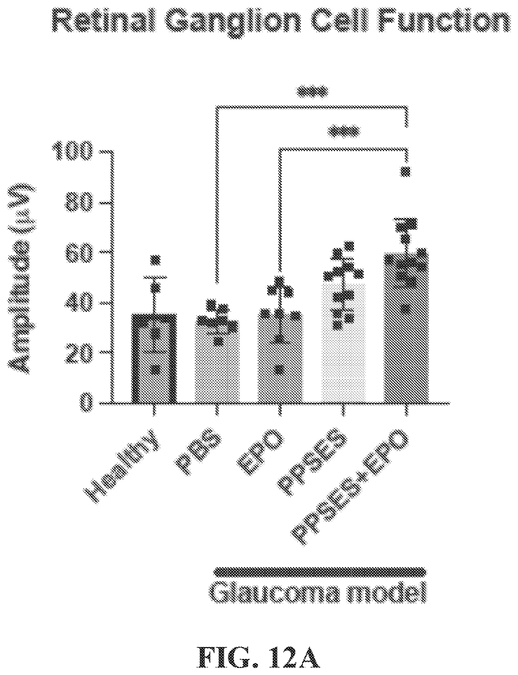

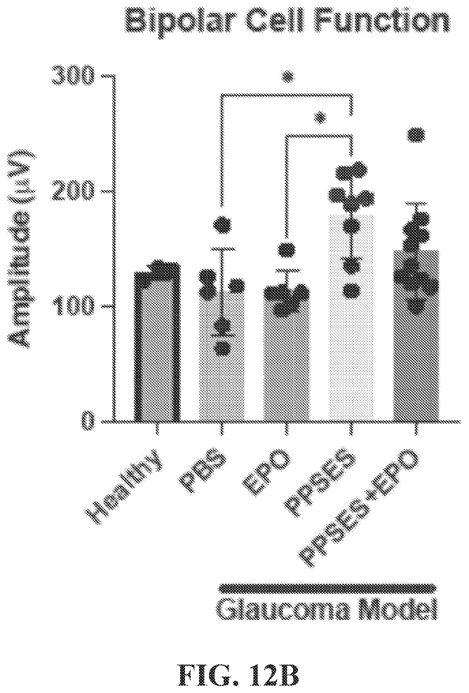

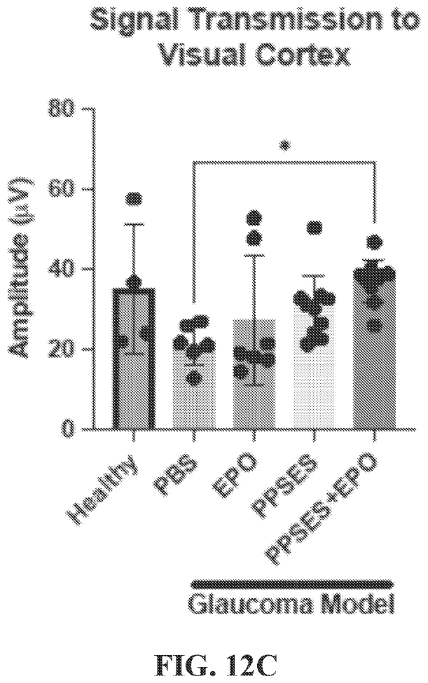

ERGs measure the function of the retina (FIG. 12A and FIG. 12B), with the B wave and photopic negative response (PhNR) measuring firing of bipolar cells and retinal ganglion cells, respectively. Notably, these metrics can be affected by multiple injections, resulting in low readings even when saline is used in healthy animals. PPSES-EPO particles increased PhNR while PPSES MPs alone showed a trend toward benefit. Similarly, Bₘₐₓ was increased with PPSES MPs, presumably due to their antioxidant activity, with an upward trend for PPSES-EPO. Finally, VEP (FIG. 12C) N1-wave amplitude, which measures ganglion cell transmission from the optic nerve to the visual cortex, was improved with PPSES-EPO treatment compared to PBS.

In summary, it was observed that the sustained release of EPO from antioxidant MPs reduced retinal ROS levels and improved visual function over EPO alone in a glaucoma model.

### Example 3

### siRNA Loaded Microparticles

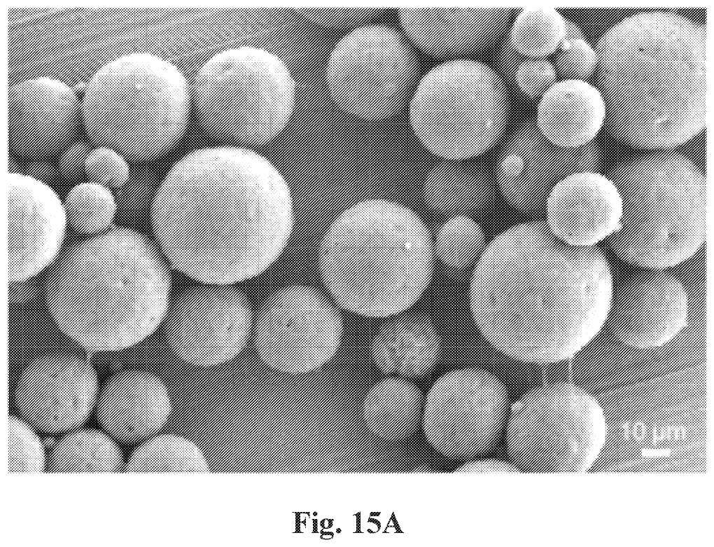

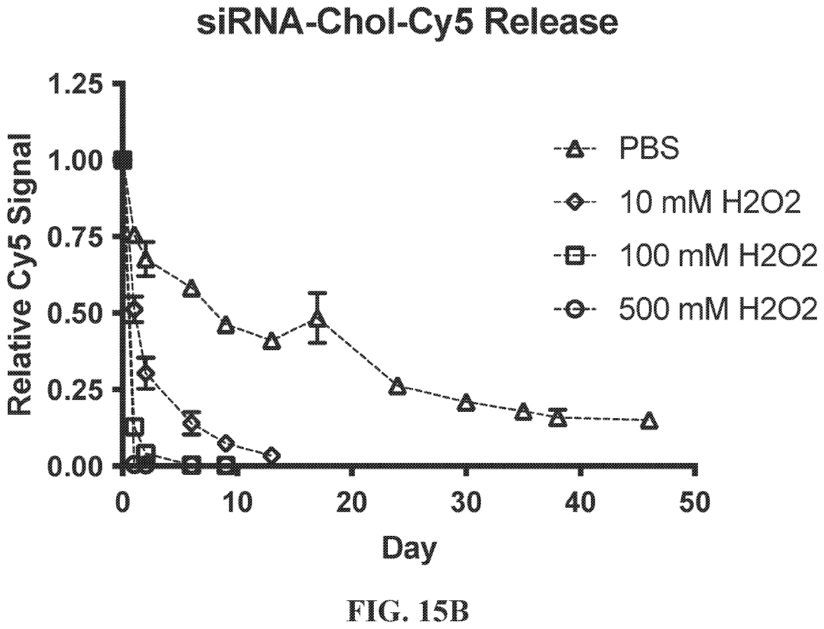

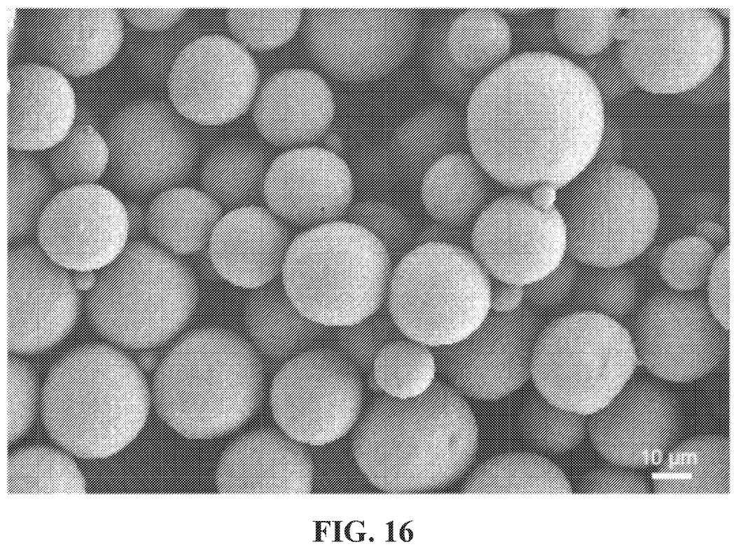

Microparticles were investigated for their ability to load oligonucleotides, such as siRNA. SiRNA in either the blunt-ended form or amphiphilic conjugates (i.e. cholesterol conjugated (FIG. 15A) or PEG-hydrocarbon conjugated (FIG. 16)) were loaded into microparticles using (1) the water-in-oil-in-water (W/O/W) method or (2) the solid-in-oil-in-water (S/O/W) method. For W/O/W, siRNA was dissolved in saline and emulsified in a polymer solution to form the W/O primary emulsion, before secondary emulsification in surfactant. siRNA:polymer mass ratio ranged from 1:100 to 1:400. For S/O/W, siRNA was lyophilized to a powder. Organic polymer solution was added to suspend the siRNA, followed by emulsification in surfactant. siRNA:polymer mass ratio was 1:100.

SiRNA was also assessed for release from the microparticle (FIG. 15B). Microparticles were loaded with Cy5-tagged siRNA. Samples were suspended in PBS or various concentrations of H₂O₂ and deposited in black-walled 96-well plates. Fluorescence images were taken at day 0 and at each relevant time point. The releasate was removed and replaced immediately prior to imaging, and the data was normalized to the fluorescent signal on day 0.

### Example 4

### Effect of Salt on EPO Microparticle Formulation

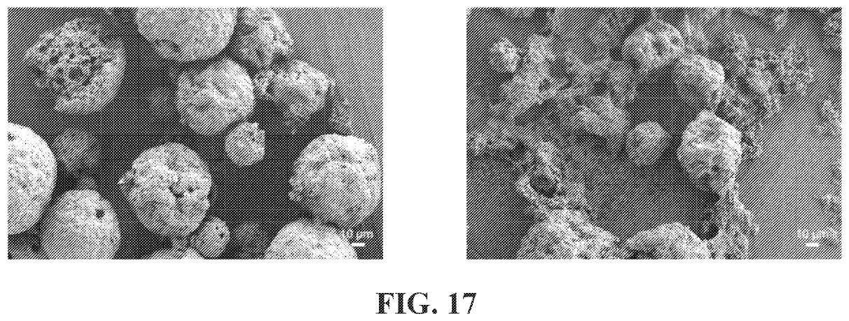

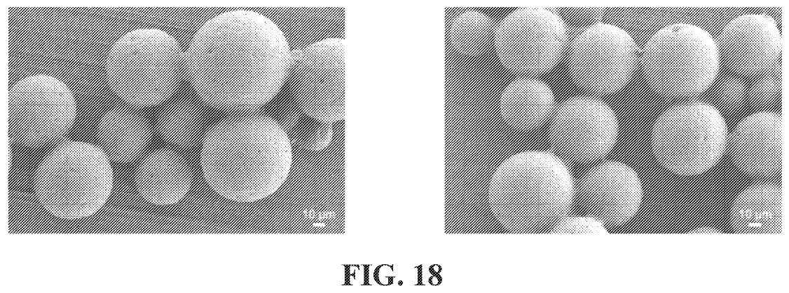

Microparticle formulation was investigated as it related to salt concentration. Commercial EPO was purchased in lyophilized format. In “high salt” formulations, the powder was reconstituted at ˜20 mg/ml, including the commercially-available salts, resulting in an osmolality of up to 56,000 mOsm/kg. After formulation into dextran nanoparticles and S/O/W formulation in PPSES microparticles, it can be seen by SEM that this large osmotic gradient from inside to outside the microparticle caused morphological disruption in the form of pore formation and dimpling (FIG. 17). In other iterations, commercial EPO was reconstituted at ˜100 μg/ml and filtered by centrifugation to concentrate the protein while allowing for free flow of the present salts, resulting in a formulation at roughly isotonic conditions (280-315 mOsm/kg) and improved subsequent microparticle morphology (FIG. 18). According, for EPO, less than 56,000 mOsm/kg can be used, such as about 100 mOsm/kg to about 1,000 mOsm/kg.

TABLE 1
Protein Loading in Microparticles

| Protein Cargo | EE (%) | Loading (w/w) |
|---|---|---|
| BSA | 80 | 0.8 |
| EPO (high salt) | 8 | 0.08 |
| EPO (low salt) | 85-95 | 0.85-0.95 |

### Example 5

### X-Ray Diffractive Spectroscopy

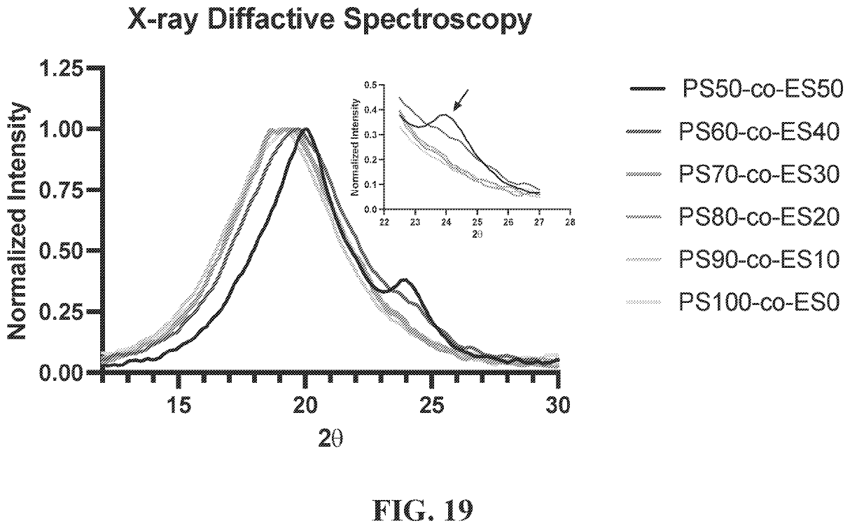

Polymers were further investigated for crystallinity using x-ray diffraction methods. Polymers were loaded into the sample holder and underwent a 0-20 diffraction scan with 20 values from 5-90° on a Rigaku Smart Lab X-ray diffraction instrument. Origin software was used to measure the area under the curve (AUC) for the crystalline (2θ˜24°) and amorphous (2θ˜20°) contributions (see R. A. Pérez-Camargo, et al. Macromolecules 2019, which is incorporated by reference herein in its entirety). Percent crystallinity (Xc, %) represents the AUC for the crystalline peak over the total signal. For polymers below 40% ES, a crystalline peak and/or shoulder was not detected, in agreement with DSC results. The results can be seen in FIG. 19 and Table 2 below.

TABLE 2
X-ray diffractive spectroscopy results

| Composition | Xc, % |
|---|---|
| PS50-co-ES50 | 13.0 |
| PS60-co-ES40 | 7.1 |
| PS70-co-ES30 | N/A |
| PS80-co-ES20 | N/A |
| PS90-co-ES10 | N/A |
| PS100-co-ES0 | N/A |

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, the following embodiments are provided.
Clause 1. A microparticle comprising: a polymer including recurring units of formula (I)

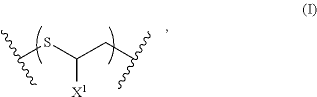

wherein: X¹ is H or CH₃; the polymer includes recurring units of X¹ is CH₃ at about 1 mol % to about 70 mol % of the polymer; the polymer includes recurring units of X¹ is H at greater than or equal to 30 mol % of the polymer; and the microparticle has a diameter of greater than 5 μm.
Clause 2. The microparticle of clause 1, wherein the polymer includes recurring units of X¹ is H at about 30 mol % to about 50 mol % of the polymer.
Clause 3. The microparticle of clause 1 or 2, wherein the microparticle has a diameter of about 5 μm to about 500 μm.
Clause 4. The microparticle of any one of clauses 1-3, wherein the polymer has a crystallinity of greater than or equal to 1% as measured by X-ray diffractive spectroscopy.
Clause 5. The microparticle of any one of clauses 1-4, wherein the recurring unit of X¹ is CH₃ is repeated 25 to 2,000 times.
Clause 6. The microparticle of any one of clauses 1-5, wherein the recurring unit of X¹ is His repeated 5 to 1,000 times.
Clause 7. The microparticle of any one of clauses 1-6, wherein the polymer has a number average molecular weight of about 1,000 Da to about 200,000 Da.
Clause 8. The microparticle of any one of clauses 1-7, wherein the polymer is a random copolymer.
Clause 9. The microparticle of any one of clauses 1-8, wherein the polymer further includes recurring units of formula (III):

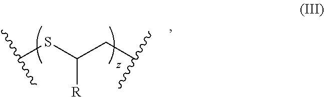

wherein: R is-C₁₋₂alkylene-R¹¹, —C₁₋₂alkylene-OC₁₋₂alkylene-R¹¹, or phenyl; R¹¹ is G¹, C₁₋₄alkyl, C₃₋₄cycloalkyl, cyano, —OH, —OC₁₋₄alkyl, —OSi(C₁₋₄alkyl) 3, —OC₁₋₂haloalkyl, —OC₃₋₄cycloalkyl, —C(O) C₁₋₄alkyl, or —CO₂C₁₋₄alkyl; G¹ is phenyl, wherein G¹ is optionally substituted with 1 or 2 substituents, each independently halogen, cyano, C₁₋₄alkyl, C₁₋₂fluoroalkyl, —OC₁₋₂alkyl, or —OC₁₋₂fluoroalkyl; and z is about 1 mol % to about 60 mol % of the polymer.
Clause 10. The microparticle of clause 9, wherein: R is-C₁₋₂alkylene-R¹¹ or phenyl; R¹¹ is G¹, C₁₋₄alkyl, or —OH; and G¹ is phenyl, wherein G¹ is optionally substituted with 1 or 2 substituents, each independently halogen, C₁₋₄alkyl, or —OC₁₋₂alkyl.
Clause 11. The microparticle of any one of clauses 1-10, further comprising a biologically active agent.
Clause 12. The microparticle of clause 11, wherein the biologically active agent comprises at least one of an enzyme, an organic catalyst, an antibiotic, an antioxidant, an anti-reactive oxygen species (ROS) agent, an anti-inflammatory, a protein, a glycoprotein, a peptide, a polyamino acid, a nucleic acid, a steroidal molecule, an antiviral, an antirejection agent, an immunosuppressant, a carbohydrate, a pharmaceutical, a cell, a virus, a virus vector, an anti-proliferative agent, an anti-migratory agent, a biologically active polymer, and a combination thereof.
Clause 13. The microparticle of clause 11 or 12, wherein the biologically active agent comprises a cytokine, a RNA, an antibody, or a combination thereof.
Clause 14. The microparticle of any one of clauses 11-13, wherein the biologically active agent comprises erythropoietin or siRNA.
Clause 15. The microparticle of any one of clauses 11-14, wherein the microparticle includes the biologically active agent at about 0.05% to about 2% by weight of the microparticle.
Clause 16. A method of treating an inflammatory disease in a subject in need thereof, the method comprising administering to the subject an effective amount of the microparticle according to any one of clauses 1-15, optionally in combination with a pharmaceutically acceptable excipient.
Clause 17. The method of clause 16, wherein the inflammatory disease is glaucoma, osteoarthritis, rheumatoid arthritis, psoriatic arthritis, pancreatitis, multiple sclerosis, cancer, diabetes, traumatic brain injury, fibrotic lung disease, atherosclerosis, ischemia-reperfusion injury, stroke, autoimmune disease, autism, macular degeneration, indirect traumatic optic neuropathy, or muscular dystrophy.
Clause 18. The method of clause 17, wherein the inflammatory disease is glaucoma or osteoarthritis.
Clause 19. The method of any one of clauses 16-18, wherein the pharmaceutically acceptable excipient comprises saline, albumin, dimethyl sulfoxide, trehalose, sucrose, polyethylene glycol, polyvinyl alcohol, pluronics, carboxymethylcellulose, an absorption enhancer, or a combination thereof.
Clause 20. The method of any one of clauses 16-19, wherein the subject is human.
Clause 21. A method of making a polymer, the method comprising: adding a first sulfide monomer and a second sulfide monomer to a reaction mixture via a syringe pump at less than or equal to 15 μl/minute, wherein the first sulfide monomer and the second sulfide monomer react to provide a random copolymer that is about 1% to about 40% crystalline.
Clause 22. The method of clause 21, wherein the first monomer is propylene sulfide and the second monomer is ethylene sulfide.
Clause 23. A method of making a microparticle, the method comprising: adding a nanoparticle comprising a dextran or other polymer and a biologically active agent to a first solvent to provide a mixture, wherein the first solvent comprises a polymer including recurring units of formula (I)

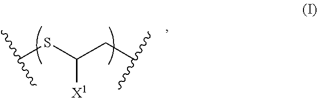

wherein: X¹ is H or CH₃, the polymer includes recurring units of X¹ is CH₃ at about 1 mol % to about 70 mol % of the polymer, and the polymer includes recurring units of X¹ is H at greater than or equal to 30 mol % of the polymer; and adding a second solvent to the mixture and emulsifying the mixture to provide a microparticle comprising the polymer and the nanoparticle.
Clause 24. The method of clause 23, wherein the dextran is cationic dextran.
Clause 25. The method of clause 23 or 24, wherein the microparticle includes dextran at about 1% to about 20% by weight of the microparticle.

What is claimed is:

1. A microparticle comprising:
a polymer including recurring units of formula (I) 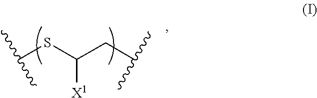
wherein:
X¹ is H or CH₃;
the polymer includes recurring units of X¹ is CH₃ at about 1 mol % to about 70 mol % of the polymer;
the polymer includes recurring units of X¹ is H at greater than or equal to 30 mol % of the polymer; and
the microparticle has a diameter of greater than 5 μm.

2. The microparticle of claim 1, wherein the polymer includes recurring units of X¹ is H at about 30 mol % to about 50 mol % of the polymer.

3. The microparticle of claim 1, wherein the microparticle has a diameter of about 5 μm to about 500 μm.

4. The microparticle of claim 1, wherein the polymer has a crystallinity of greater than or equal to 1% as measured by X-ray diffractive spectroscopy.

5. The microparticle of claim 1, wherein the recurring unit of X¹ is CH₃ is repeated 25 to 2,000 times.

6. The microparticle of claim 1, wherein the recurring unit of X¹ is H is repeated 5 to 1,000 times.

7. The microparticle of claim 1, wherein the polymer has a number average molecular weight of about 1,000 Da to about 200,000 Da.

8. The microparticle of claim 1, wherein the polymer is a random copolymer.

9. The microparticle of claim 1, wherein the polymer further includes recurring units of formula (III): 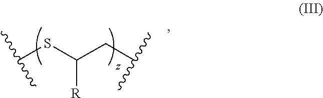
wherein:
R is —C₁₋₂alkylene-R¹¹, —C₁₋₂alkylene-OC₁₋₂alkylene-R¹¹, or phenyl;
R¹¹ is G¹, C₁₋₄alkyl, C₃₋₄cycloalkyl, cyano, —OH, —OC₁₋₄alkyl, —OSi(C₁₋₄alkyl) 3, —OC₁₋₂haloalkyl, —OC₃₋₄cycloalkyl, —C(O) C₁₋₄alkyl, or —CO₂C₁₋₄alkyl;
G¹ is phenyl, wherein G¹ is optionally substituted with 1 or 2 substituents, each independently halogen, cyano, C₁₋₄alkyl, C₁₋₂fluoroalkyl, —OC₁₋₂alkyl, or —OC₁₋₂fluoroalkyl; and
z is about 1 mol % to about 60 mol % of the polymer.

10. The microparticle of claim 9, wherein:
R is —C₁₋₂alkylene-R¹¹ or phenyl;
R¹¹ is G¹, C₁₋₄alkyl, or —OH; and
G¹ is phenyl, wherein G¹ is optionally substituted with 1 or 2 substituents, each independently halogen, C₁₋₄alkyl, or —OC₁₋₂alkyl.

11. The microparticle of claim 1, further comprising a biologically active agent.

12. The microparticle of claim 11, wherein the biologically active agent comprises at least one of an enzyme, an organic catalyst, an antibiotic, an antioxidant, an anti-reactive oxygen species (ROS) agent, an anti-inflammatory, a protein, a glycoprotein, a peptide, a polyamino acid, a nucleic acid, a steroidal molecule, an antiviral, an antirejection agent, an immunosuppressant, a carbohydrate, a pharmaceutical, a cell, a virus, a virus vector, an anti-proliferative agent, an anti-migratory agent, a biologically active polymer, and a combination thereof.

13. The microparticle of claim 11, wherein the biologically active agent comprises a cytokine, a RNA, an antibody, or a combination thereof.

14. The microparticle of claim 11, wherein the biologically active agent comprises erythropoietin or siRNA.

15. The microparticle of claim 11, wherein the microparticle includes the biologically active agent at about 0.05% to about 2% by weight of the microparticle.

16. A method of treating an inflammatory disease in a subject in need thereof, the method comprising administering to the subject an effective amount of the microparticle according to claim 1, optionally in combination with a pharmaceutically acceptable excipient.

17. The method of claim 16, wherein the inflammatory disease is glaucoma, osteoarthritis, rheumatoid arthritis, psoriatic arthritis, pancreatitis, multiple sclerosis, cancer, diabetes, traumatic brain injury, fibrotic lung disease, atherosclerosis, ischemia-reperfusion injury, stroke, autoimmune disease, autism, macular degeneration, indirect traumatic optic neuropathy, or muscular dystrophy.

18. The method of claim 17, wherein the inflammatory disease is glaucoma or osteoarthritis.

19. The method of claim 16, wherein the pharmaceutically acceptable excipient comprises saline, albumin, dimethyl sulfoxide, trehalose, sucrose, polyethylene glycol, polyvinyl alcohol, pluronics, carboxymethylcellulose, an absorption enhancer, or a combination thereof.

20. The method of claim 16, wherein the subject is human.

21. A method of making a polymer, the method comprising:
adding a first sulfide monomer and a second sulfide monomer to a reaction mixture via a syringe pump at less than or equal to 15 μl/minute, wherein the first sulfide monomer and the second sulfide monomer react to provide a random copolymer that is about 1% to about 40% crystalline as measured by X-ray diffractive spectroscopy.

22. The method of claim 21, wherein the first monomer is propylene sulfide and the second monomer is ethylene sulfide.

23. A method of making a microparticle, the method comprising:
adding a nanoparticle comprising a dextran or other polymer and a biologically active agent to a first solvent to provide a mixture, wherein the first solvent comprises a polymer including recurring units of formula (I) 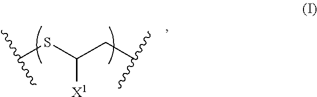
wherein:
X¹ is H or CH₃,
the polymer includes recurring units of X¹ is CH₃ at about 1 mol % to about 70 mol % of the polymer, and
the polymer includes recurring units of X¹ is H at greater than or equal to 30 mol % of the polymer; and
adding a second solvent to the mixture and emulsifying the mixture to provide a microparticle comprising the polymer and the nanoparticle.

24. The method of claim 23, wherein the dextran is cationic dextran.

25. The method of claim 23, wherein the microparticle includes dextran at about 1% to about 20% by weight of the microparticle.
